(19) United States
(12) Patent Application Publication
(10) Pub. No.: US 2026/0207137 A1
(43) Pub. Date: Jul. 23, 2026

(54) Field-adaptable, Functionalized Textile for Battery-free Body Area Networks

(71) Applicant: The Regents of the University of California, Oakland, CA (US)

(72) Inventors: Peter Tseng, Irvine, CA (US); Amirhossein Hajiaghajani, Irvine, CA (US); Huiting Qin, Irvine, CA (US); Abel Jimenez, Irvine, CA (US)

(21) Appl. No.: 19/141,289

(22) PCT Filed: Dec. 22, 2023
(86) PCT No.: PCT/US2023/085793

§ 371 Date: Jun. 19, 2025

Related U.S. Application Data
(60) Provisional application No. 63/436,832, filed on Jan. 3, 2023.

(51) Int. Cl. A61B 5/00; A41D 13/12; A61B 5/0205; A61B 5/021; A61B 5/024; A61B 5/08; A61B 5/27; A61B 5/318; A61B 5/389; H04B 5/24

(52) U.S. Cl. CPC A61B 5/6804; A41D 13/1281; A61B 5/0205; A61B 5/27; H04B 5/24; A61B 5/021; A61B 5/02416; A61B 5/0816; A61B 5/318; A61B 5/389; A61B 2503/06

(57) ABSTRACT

Field-adaptable, functionalized textiles for body area networks in accordance with embodiments of the invention are disclosed. In one embodiment, a textile body area network is provided, the textile body area network comprising: a wireless reader; a first smart textile configured to perform cross-body measurements, the first smart textile comprising: a first array of magnetically coupled resonators configured to propagate magnetoinductive (MI) surface waves; and at least one first sensor configured to connect to the first array of magnetically coupled resonators, wherein the at least one first sensor generates an output signal based on a physical measurement.

### CROSS REFERENCE TO RELATED APPLICATION

The current application claims priority to U.S. Provisional Patent Application No. 63/436,832 filed on Jan. 3, 2023, the disclosure of which is incorporated herein by reference.

### FEDERAL FUNDING SUPPORT

This invention was made with Government support under Grant No. ECCS-1942364, awarded by the National Science Foundation. The Government has certain rights in the invention.

### FIELD OF THE INVENTION

The present invention generally relates to wireless communications and more specifically to body area networks.

### BACKGROUND

Fast-growing human health-monitoring and activity-tracking technologies are poised to revolutionize our day-to-day lives. Facilitating these modern tools are an ever-growing number of wearable or implantable, physical or analytical sensors that link to disparate regions of the body and/or link multiple bodies. Such multi-node networks may parse information from complex systems such as our bodies and objects that may be interacted with. Typically, these emerging technologies necessitate robust, secure, and reliable communication links between nodes in order to parse biometric information in real-time. Such links are generally known as body area networks (BANs), and are potential building blocks of emerging technologies such as, but not limited to, health care monitoring.

### SUMMARY OF THE INVENTION

The various embodiments of the present field-adaptable, functionalized textiles for battery-free BANs (may also be referred to herein as “textile-BANs”) contain several features, no single one of which is solely responsible for their desirable attributes. Without limiting the scope of the present embodiments, their more prominent features will now be discussed below. In particular, the present field-adaptable, functionalized textiles for battery-free BANs will be discussed in the context of monitoring congenital heart disease (CHD). However, the use of functionalized textiles for monitoring CHD is merely exemplary and the present approach and embodiments may scale, adapt, and generalize to many conditions that require full-body monitoring. Further, the present functionalized textiles will be discussed in the context of particular sensors (e.g., NFC sensors, biosensors, etc.). However, use of particular sensors are also merely exemplary and various other sensors utilizing particular communication protocols and/or for detecting and/or measuring a variety of physical properties may be utilized for field-adaptable, functionalized textiles for battery-free BANs as appropriate to the requirements of a specific application in accordance with various embodiments of the invention. After considering this discussion, and particularly after reading the section entitled “Detailed Description,” one will understand how the features of the present embodiments provide the advantages described here.

In a first aspect, a textile body area network is provided, the textile body area network comprising: a wireless reader; a first smart textile configured to perform cross-body measurements, the first smart textile comprising: a first array of magnetically coupled resonators configured to propagate magnetoinductive (MI) surface waves; and at least one first sensor configured to connect to the first array of magnetically coupled resonators, wherein the at least one first sensor generates an output signal based on a physical measurement.

In an embodiment of the first aspect, the first array of magnetically coupled resonators comprises a first plurality of MI elements; and the first array of magnetically coupled resonators creates a first flexible magnetic metamaterial path for wireless communication using the MI surface waves.

In another embodiment of the first aspect, the at least one first sensor is a laser-induced graphene sensor.

In another embodiment of the first aspect, the at least one first sensor is a strain sensor that measures motion and respiration.

In another embodiment of the first aspect, the at least one first sensor is a photoplethysmography (PPG) sensor that measures circulation, pulse transit, and blood pressure.

In another embodiment of the first aspect, the at least one first sensor is a bioelectricity sensor for electromyography (EMG) and electrocardiogram (ECG).

In another embodiment of the first aspect, the first smart textile is constructed by heat pressing the at least one first sensor to a first article of clothing, overlapping a textile system-patch with the at least one first sensor, and sewing the textile system-patch to the first article of clothing.

In another embodiment of the first aspect, the first smart textile is constructed by attaching the at least one first sensor to a textile system-patch and sewing the textile system-patch to a first article of clothing.

In another embodiment of the first aspect, the first smart textile is constructed by adhering the at least one first sensor to a first article of clothing and adhering a textile system-patch to the first article of clothing.

In another embodiment of the first aspect, the first smart textile is constructed using a compression strap and an external adjustable strap.

In another embodiment of the first aspect, the field adaptable network further comprises: a second smart textile configured to perform cross-body measurements, the second smart textile comprising: a second array of magnetically coupled resonators configured to propagate MI surface waves; and at least one second sensor configured to connect to the second array of magnetically coupled resonators, wherein the at least one second sensor generates an output signal based on a physical measurement.

In another embodiment of the first aspect, the second array of magnetically coupled resonators comprises a second plurality of MI elements; and the second array of magnetically coupled resonators creates a second flexible magnetic metamaterial path for wireless communication using the MI surface waves.

In another embodiment of the first aspect, the at least one second sensor is a laser-induced graphene sensor.

In another embodiment of the first aspect, the at least one second sensor is a strain sensor that measures motion and respiration.

In another embodiment of the first aspect, the at least one second sensor is a PPG sensor that measures circulation, pulse transit, and blood pressure.

In another embodiment of the first aspect, the at least one second sensor is a bioelectricity sensor for EMG and ECG.

In another embodiment of the first aspect, the second smart textile is constructed by heat pressing the at least one second sensor to a second article of clothing, overlapping a textile system-patch with the at least one second sensor, and sewing the textile system-patch to the second article of clothing.

In another embodiment of the first aspect, the second smart textile is constructed by attaching the at least one second sensor to a textile system-patch and sewing the textile system-patch to a second article of clothing.

In another embodiment of the first aspect, the second smart textile is constructed by adhering the at least one second sensor to a second article of clothing and adhering a textile system-patch to the second article of clothing.

In another embodiment of the first aspect, the second smart textile is constructed using a compression strap and an external adjustable strap.

In another embodiment of the first aspect, the first smart textile and the second smart textile are battery-free.

In another embodiment of the first aspect, the first article of clothing is a long shirt and the second article of clothing is a pants.

In another embodiment of the first aspect, the first smart textile and the second smart textile are separated by a clothing transition.

In another embodiment of the first aspect, the MI surface waves generate a wireless communication link across the clothing transition.

In another embodiment of the first aspect, the wireless reader performs edge-based AI for learning, identification, and power-handling decisions.

### BRIEF DESCRIPTION OF THE DRAWINGS

The various embodiments of the present field-adaptable, functionalized textiles for BANs now will be discussed in detail with an emphasis on highlighting the advantageous features. These embodiments depict the novel and non-obvious features of field-adaptable, functionalized textiles for BANs shown in the accompanying drawings, which are for illustrative purposes only. These drawings include the following figures:

FIG. 1 is a diagram illustrating edge-AI and BAN for monitoring pediatric CHD in accordance with certain embodiments of the invention.

FIG. 2a is a schematic diagram illustrating a planar magnet resonator in accordance with certain embodiments of the invention.

FIG. 2b are graphs illustrating a ground layer minimizing spectral uncertainty due to a human body's parasitic effect in accordance with certain embodiments of the invention.

FIG. 2c is a schematic diagram illustrating an array of magnetically coupled resonators with a horizontal distance (in x direction) and a vertical distance in other directions in accordance with certain embodiments of the invention.

FIG. 2d illustrates an equivalent circuit of the magnetoinductive (MI) metamaterial with potential object-to-object transitions in accordance with certain embodiments of the invention.

FIG. 2e are dispersion diagrams for an array of resonators for various magnetic coupling coefficients in accordance with certain embodiments of the invention.

FIG. 2f are diagrams illustrating a reader and multiple sensors utilized in an in-line serial and a T-shaped parallel array of resonators to form various signal paths around a body in accordance with certain embodiments of the invention.

FIG. 2g is a diagram illustrating a metamaterial network (may also be referred to as “body area network” or “BAN”) streamlined into separate clothing pieces in accordance with certain embodiments of the invention.

FIG. 3a illustrates a high speed, long-term indoor walk/running activity measurement in accordance with certain embodiments of the invention.

FIG. 3b illustrates monitoring of sensors during indoor running under various velocity profiles in accordance with certain embodiments of the invention.

FIG. 4a is a diagram illustrating a jumper element in accordance with certain embodiments of the invention.

FIG. 4b is a graph illustrating a multi-band network transmission profile for inter-coil (horizontal) coupling versus vertical coupling generated by a vertical distance in the middle of the network in accordance with certain embodiments of the invention.

FIG. 4c illustrates a multi-passband metamaterial element showing active excitation, muscle tissue, magneto-inductive waveguide, and a passive reader illustrating SAR at 1 W in accordance with certain embodiments of the invention.

FIG. 5a is a diagram illustrating textile sensors based on laser-machined graphene in accordance with certain embodiments of the invention.

FIG. 5b are graphs illustrating differential response of strain sensors to motion in accordance with certain embodiments of the invention.

FIG. 6a is a diagram illustrating a multisensor wristband for neonates in accordance with certain embodiments of the invention.

FIG. 6b are graphs illustrating machine learning (ML) on a multisensory device for intake monitoring in accordance with certain embodiments of the invention.

FIG. 7 is a diagram illustrating BAN layouts for CHD in accordance with certain embodiments of the invention.

FIGS. 8a-c are diagrams illustrating advanced magnetic metamaterials for augmented BANs in accordance with certain embodiments of the invention.

FIG. 9 is a diagram illustrating figures of merit (FOM) in accordance with certain embodiments of the invention.

FIG. 10 is a diagram illustrating permanent or reconfigurable systems integration into textiles in accordance with certain embodiments of the invention.

FIG. 11 is a diagram illustrating textile stabilization techniques in accordance with certain embodiments of the invention.

FIG. 12 is a diagram illustrating a domain agnostic framework in accordance with certain embodiments of the invention.

FIG. 13 is a diagram illustrating a particle swarm optimization framework in accordance with certain embodiments of the invention.

FIGS. 14a-b are graphs illustrating application of a laser-induced graphene (LIG) textile for pulse detecting in accordance with certain embodiments of the invention.

FIGS. 15a-d illustrate a graphene-based (e.g., LIG) multifunctional textile for integrated sensing platforms in accordance with certain embodiments of the invention in accordance with certain embodiments of the invention.

FIGS. 16a-f illustrate mechanical performance of an LIG when used as a strain sensor in accordance with certain embodiments of the invention.

FIGS. 17a-i illustrate different human motions detected by a LIG used as a strain sensor in accordance with certain embodiments of the invention.

FIGS. 18a-j illustrate design and characterization of an LIG-based humidity and temperature sensor in accordance with certain embodiments of the invention.

### DETAILED DESCRIPTION OF THE DRAWINGS

The following detailed description describes the present embodiments with reference to the drawings. In the drawings, reference numbers label elements of the present embodiments. These reference numbers are reproduced below in connection with the discussion of the corresponding drawing features.

Point-of-care, wearable or implantable devices are an important technology class within precision health. For example, device-enabled, real-time monitoring of biometrics provides useful feedback on personal health and early warnings on poor body function. Significant research effort has been placed on sensing technologies and body-interfacing microsystems that potentially distribute around (and within) the body while collecting local biomechanical and/or chemical information. It is anticipated that networks of such sensors (may also be referred to herein as BANs) can transform understanding of human health through integrated body-distributed biometrics. Ultimately, such networks are natural intelligence gateways into human bodies that are hierarchical and exhibit significant spatial complexity. In combination with modern approaches in data analysis and decision-making, BANs could impact multiple scales of decision-making in health, ranging from enhancing individual and/or caretaker choices to improving medical and governmental interventions.

One potential application for these technologies is in daily monitoring of those afflicted with congenital heart disease (CHD), or those born with one or more defects in the heart. Close to 3 million are estimated to have this condition in the US, and a large percentage are children who face short life expectancies. This disease practically manifests as across-body variation in blood pressure and oxygenation (i.e., coarctation, cyanosis, etc.), as well as person-to-person impacts on heart rate. One clinical sign of CHD is lower oxygen saturation (SpO2) in legs than in arms. Thus, precision medicine is a powerful approach to treat CHD, and in particular BANs may be necessary to improve understanding of daily health, rapidly identify complications, continuously track stress response, and monitor kids during exercise routines (fitness improves CHD symptoms). BANs are uniquely suited to CHD as multi-site and fully-body monitoring is necessary to better understand daily stressors (e.g., movements and/or exercises that strain the heart), and for accurate estimation of key circulation biometrics such as oxygenation in distal points, pulse transit, and blood pressure variation across body. Biometrics could impact multiple scales of decision-making in CHD, enhancing personal choices or improving interventions. Integrated edge-AI could be used to both analyze data and direct simple interventions. Finally, distributed sensor networks could be further field-adapted to respective patients' needs and monitor other ailments or bodily processes.

The present embodiments include several approaches to directly embed sensors, networks, and systems into textile to form field adaptable, battery-free body area networks on clothing. In many embodiments, sensors and networks are embedded directly into textiles using multiple layers of material heat-pressing/layering and engineering. Further, the present embodiments include textile sensors that may include engineering of layers to achieve selectivity and functionality. In addition, the present embodiments include techniques to embed systems either permanently or temporarily (for field adaptability). For example, systems-patches may be embedded inside patches, which are adhered to clothing using sewing, Velcro, etc. In some embodiments, integrated strap elements can further assist to seal sensors against the skin.

The present embodiments generate easy-to-use, low burden BANs for various purposes in body tracking, biometric monitoring, pain management, etc. As further described below, the BANs are field-adaptive as the sensors and systems can be built and shifted by the requirements of a particular application. Further, because the various components are built into textiles (e.g., clothing), the application and use of such functionalized textiles may reduce complications and burdens associated with every day use. In addition, the battery-free nature also provides benefits as the functionalized textiles can scale in complexity without burdening the user.

In addition, the present embodiments combine smart textiles and edge-driven ML techniques to create patient-focused, secure human body-learning networks. New fabrication and integration approaches are considered for the on-demand fusion of textiles, sensors, and electronics into usable wearable platforms. In many embodiments, textile-borne magnetic metamaterials may be utilized as a flexible approach for powering wireless wearable and implantable sensing nodes with consideration of human body-focused electromagnetics simulation and development. In various embodiments, unique biometric, edge-ML computation may be adapted to variable networks of wearable sensing nodes that change in position, number, and power while biometric data may shift in domain. Validation of monitoring networks on pediatric subjects are further described below.

### Validation of Field-Adaptable, Functionalized Textiles for BANs on Pediatric Subjects

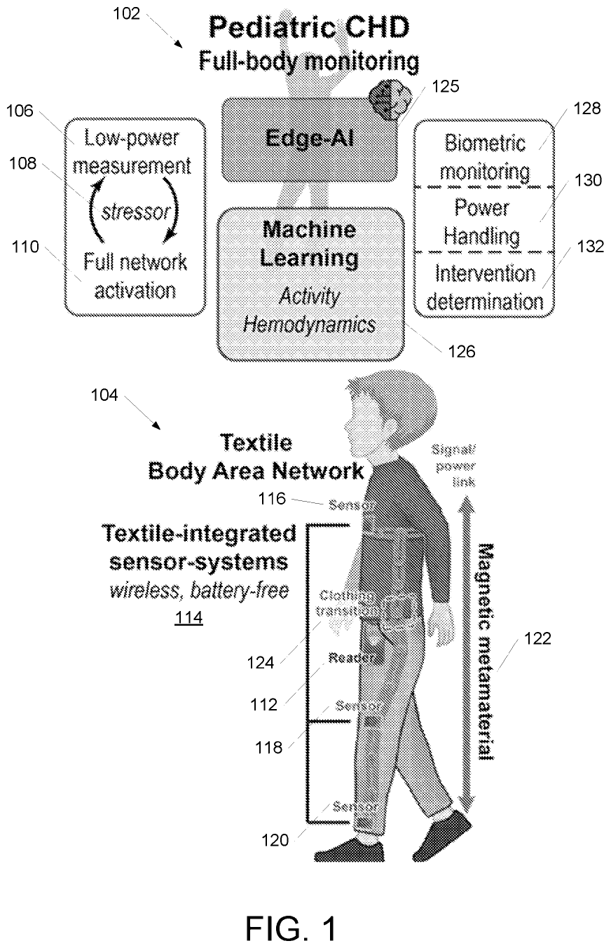

Field-adaptable, functionalized textiles may be utilized for BANs (may also be referred to herein as “textile body area networks”). In particular, such monitoring systems may be utilized with pediatric subjects. FIG. 1 is a diagram illustrating edge-AI 102 and BAN 104 for monitoring pediatric CHD in accordance with certain embodiments of the invention. A daily monitoring network for kids with CHD is illustrated. Due to power considerations, the system may perform intermittent, low-power measurement 106 until the user encounters a stressor 108, at which point the network activates to perform full body monitoring 110 of biometrics during key events such as, but not limited to, walking upstairs, running, etc. In many embodiments, the base system may be implemented by two distinct, wearable technological components: (a) a single, battery-powered wireless reader 112 that serves as the edge-AI head of the network while performing key learning, identification, and power-handling decisions, and (b) a battery-free, smart textile with an embedded wireless-BAN 114 (e.g., sensors 116, 118, 120) that performs advanced cross-body measurement when necessary. In certain embodiments, the base BAN may measure activity and circulation metrics, which can be captured using strain and photoplethysmography (PPG) sensors distributed across the body. In some embodiments, the BAN may also monitor various signals such as, but not limited to, bioelectric signals from the heart and certain skeletal muscles to captures cardiac electrical signals and muscular effort, respectively. In various embodiments, the smart textiles may be separated by a clothing transition 124.

In many embodiments, the smart textile is the most advanced technological component of such systems. In certain embodiments, its wireless network may be facilitated by an advanced, textile-integrated magnetic metamaterial 122 that can transmit nearfield signal intermediate distances (even across discontinuous bodies). This delivers wireless power to (and draws information from) battery-free, wireless sensor systems that also modularly integrate into clothing. This network 104 is uniquely versatile and field-adaptable. For example, the network 104 may be supportive of existing NFC or Bluetooth-based devices that may be worn anywhere close the network 104. Further, its higher-power capacity can deliver required power for controlling actuators or interacting with implantable devices. The textile may also contain embedded elements to ensure specific contact sensors are fixed against the body (e.g., straps or adhesives) and may be used in textile-borne monitors for sensors such as EMG and PPG. In some embodiments, the sensors may also include various types of perspiration sensors such as, but not limited to, sweat pH sensors, perspiration rate sensors, ion-selective sensors, etc.

In reference to FIG. 1, BANs 104 are the ideal monitoring mode for those with CHD. Cross-body, multi-site monitoring of motion or PPG is uniquely suited for accurate estimation of specific user activity/exercise stressor, pulse transit time, blood pressure, and SpO2 at extremities that may vary across the body for those with CHD. Multi-site PPG improves blood pressure and pulse transmit time estimation. Additionally, full-body measurement of motion and blood circulation may yield information on the response of the heart to stressors that cannot be captured in 1 or 2 sensor positions. Such advanced knowledge may be able to provide early warning on heart conditions, monitor heart fatigue, track body progress or response to drugs, and immediately direct field interventions such as rest or sleep.

In addition, “smart” clothing is an ideal, comfortable interface for kids as clothes are already worn every day. The present textiles can have scaling complexity with no change in user-burden. Typically, a standard approach of attaching more devices on the body quickly becomes both unmanageable and uncomfortable for the user. The present networks' battery-free, wireless sensing nodes also minimize both weight and required interactions.

The present magnetic metamaterial network is uniquely powerful in comparison to wired or alternative wireless network schemes. This network is field-adaptable to patient needs-nodes measuring a variety of relevant biometrics (utilizing NFC or Bluetooth protocols) can be placed anywhere in proximity of the metamaterial as required, and metamaterials even allow RF energy transfer seamlessly across clothing. User-burden is minimized due to zero batteries and minimal wiring/interconnects enabled by metamaterial-enabled wireless power transfer. The network exhibits resilience and minimizes failure as it does not require long-range wiring and does not have exposed leads that can mechanically-fail or are prone to degradation. Further, the present networks may scale into future network applications by interfacing with existing implantable or actuating devices, such devices will require wireless power that either penetrates tissue or can be safely distributed near the body which is uniquely enabled by RF magnetic fields that are only weakly absorbed by the body.

The present networks allow for new capabilities, optimizations, and implementations in various target groups. A focus of the present embodiments includes metamaterials, microsystems, and their fusion in textile, advanced magnetic metamaterials may be used that not only support multiple frequency bands (for enhanced control of RF power/information) but improved transmissivity. Key FOM of such approaches may be characterized allowing for on-demand methods of integrating sensors and microsystems directly into textile. As illustrated in FIG. 1, another focus of the present embodiments includes an edge-based artificial intelligence (AI) 125 that may control and monitor (e.g. biometric monitoring 128, power handling 130, intervention determination 132, etc.) the network 104, while analyzing biometric data. Machine Learning (ML) frameworks 126 may be optimized for BANs that may shift node number and domain. For example, such ML frameworks may learn each person over time, understanding activities and circulatory response.

Although specific validation of textile-BANs for pediatrics are discussed above with respect to FIG. 1, any of a variety of textile-BANs as appropriate to the requirements of a specific application can be utilized in accordance with embodiments of the invention. A technology overview in accordance with embodiments of the invention are further described below.

### Technology Overview

Wearable devices, PPG and BANs: Non-invasive, point-of-care devices have emerged as powerful tools to biometric monitoring. In this class of devices, point-of-care interfaces in the form of flexible devices and tattoos have become popular due to their low form factor, reduced user burden, and ability to monitor physiology in complex areas. An emerging subset of these devices include textile-integrated sensors that seek to provide seamless integration of sensing functionality into clothing. Textile sensors (e.g., EMG, strain, etc.) are often generated via functionalized threads, bioprinting, or heat-pressing of soft sensors onto textiles. Trickier is integration of the backend system into textile, which must sit on printed circuit board (PCB) and are often inelegant.

PPG sensors are clinically used to monitor pulse rate, oximetry (for SpO2), and blood pressure. For simple monitoring of pulse rate and blood pressure (which improves with multi-site monitoring), the PPG can be placed nearly anywhere. Oximetry is traditionally done on fingertips, earlobes, or toes for highest accuracy, however alternative locations (wrist-Apple Watch/oxitone, ankle-BSX, thigh-humon hex, upper arm-CMI Health) have been clinically validated in modern wearable devices. Due to thinner skin/less fat in kids, accuracy tends to be higher than in adults. In addition, accuracy may reduce at very low blood pressures, this should ultimately be factored in to intervention decision-making.

Modern sensor networks are traditionally composed of wired or wireless nodes of sensors that are spatially distributed while measuring environmental signals at each location. Wired networks are directly connected via electrical contacts that supply power and facilitate information transfer through wires. Wired networks can be prone to mechanical failure, corrosion of leads, safety issues from contacts, and cannot readily transfer signal across multiple clothing. Wireless nodes are either battery or wireless-powered while transmitting data over the air (via Zigbee, enOcean, WiFi, Bluetooth, NFC, etc.). A subset of low-power sensing nodes is sometimes referred to as passive devices (i.e., they do not require a battery), wherein Bluetooth or NFC are able to wirelessly supply the power required to operate the node. Battery-free Bluetooth is fairly limited as Bluetooth does not transmit much power over the air and electric fields are readily absorbed/scattered by the body and environment. NFC uses nearfield magneto-inductive powering and due to minimal absorption of RF magnetic fields by the body these can both penetrate into body tissue and be used to deliver higher power adjacent to the human body without induced unwanted tissue heating. Bluetooth and NFC have emerged as dominant protocols in the biometric realm. NFC's nearfield powering of sensors/devices has high security and is used in FDA-approved and biohacking devices ranging from implantables (e.g., Senseonics CGM), to wearables (e.g., FreeStyle Libre), to body-embedded RFID.

A recent subset of sensing networks includes BANs, that similarly can be composed of wired or wireless nodes of sensors distributed along the body. Due to the interesting parameters of the body, several notable derivatives of the wired of wireless varieties have appeared, wherein the body or skin itself can be used as a conductor, or waveguides flanking the body can be used to either power wireless nodes or facilitate information transfer. For example, spoof-plasmon metamaterials or magnetoinductive waveguides can be integrated into textiles to transmit signal.

Magnetic metamaterials: Magnetic metamaterials are a candidate technology to facilitate the delivery of RF magnetic power. These consist of arrays of inductors that can support the propagation of magnetoinductive (MI) waves along its pathway (forming a waveguide). Such MI waves exhibit comparatively low loss in attenuating media and have been proposed as an approach to spatially transport waves across soil, salt water/ocean, during MRI, and along the body. Despite its potential, many such investigations have primarily been theoretical or in small testbeds as in practice, inductors orient perpendicularly and are difficult to directly implant into environments. In addition, the functional distance of RF magnetic power transported by these waves is relatively low because the entire waveguide is homogeneous and active in the nearfield.

Machine learning and biomedical devices: With large volumes of health care datasets and advancements in deep learning techniques, systems are now potentially well-equipped in the diagnosis of many health problems. Among these deep learning models, convolutional neural networks (CNN) are often used for their automatic feature extraction and memory-based recurrent neural network (RNN) architectures such as the long short-term memory (LSTM) and gated recurrent units (GRU), have been studied for extracting temporal patterns in timeseries healthcare applications. Much of deep learning's success relies on the underlying assumption that the training (source) data and the testing (target) data are independent and identically distributed (i.i.d.), in other words, they come from the same data distribution. However, training and deployment conditions may vary drastically causing a domain shift (DS) between what was learned and what is being perceived leading to degraded model generalization performance. Within medical applications, this can be due to inter-subject variability (differences between subjects), intra-subject variability (changes in subject overtime), hardware variability (differences in devices/sensors), etc. To address this critical issue, domain adaptation techniques have been used to correct the distribution shift by minimizing the distribution differences between the target and the source data. In contrast, domain-invariant feature learning aims to find underlying latent features that can generalize to the task regardless of domain-induced shifts.

On the other hand, BANs are distributed computing structures with multiple sensor nodes where some are more energy demanding than others. Due to this characteristic, overall system uptime and utility can be bottlenecked by out of power nodes. Depending on the required performance criterions, the present embodiments can effectively manage such a system by solving a multi-objective optimization problem. Most commonly optimization problems can be solved using numeric solvers, however they are often computationally expensive, time consuming and can only be applied to static structures offline. Other approaches include game theory, maps the problem to finding Nash equilibrium in a multiagent game, clustering algorithms like particle swarm randomly iterates through possible solutions to find the global maximum or ant colony that maps the problem into solving optimal paths, and reinforcement learning to train agents to make optimal actions given state observations and a corresponding reward/penalty system.

Although specific technology for textile-BANs are discussed above, any of a variety of technologies as appropriate to the requirements of a specific application can be utilized in accordance with embodiments of the invention. A data consideration in accordance with embodiments of the invention are further described below.

### Data Considerations

This proposal performs integrative research on materials science, electromagnetism, embedded systems, and machine learning towards the integration adaptable, wireless BANs to within clothing. Our group has a wide breadth of expertise in sensing technologies (including for neonates), “smart” textiles, edge-ML, and exercise. Our medical collaborators operate the UCI Pediatric Exercise and Genomics Research Center, with experience in pediatric exercise monitoring for healthy or ill kids (perc.uci.edu). This center houses advanced cardiopulmonary testing equipment and collaborates with cardiologists, pulmonologists, etc.

Textile-integrated, magnetic metamaterials and nearfield, multi-body area networks: The present embodiments include fused magnetic metamaterials on textile to enable battery-free, multi-BANs. MI waves propagate through an array of magnetically-coupled resonant structures, in this case equivalent planar inductors. Resonators may be designed in variant forms depending on desired network behavior, and routed along the body through splitting elements. Multiturn flexible planar coils are made of metal foils (on vinyl). To remove the effect of tissue on the spectral behavior of the coil, we can either create a ground shield layer or utilize a lumped capacitance for stabilization. Structural response remained stable during mechanical distresses. We proposed a facile and versatile technique of integrating metamaterial railways on textile. Layers were stacked, placed on the clothing, and finally fixed by heat pressing.

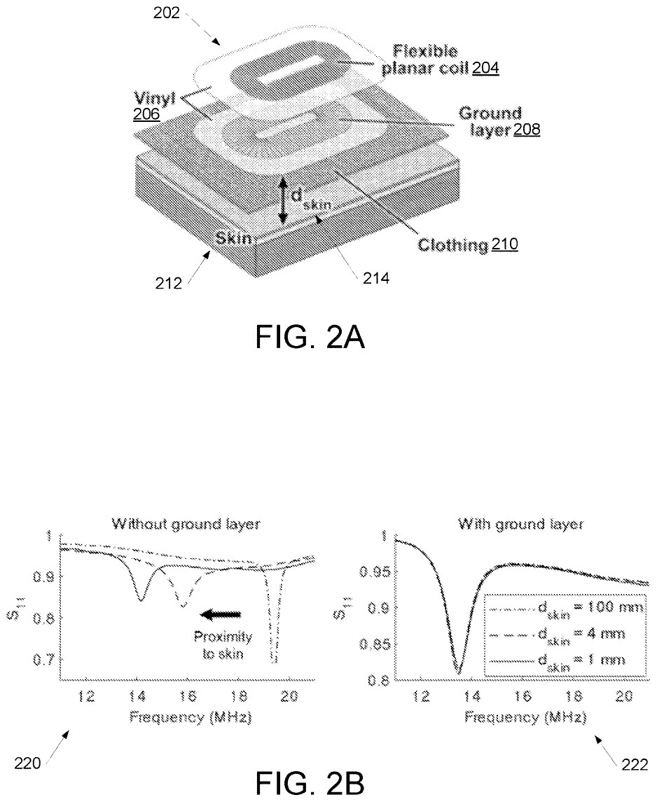

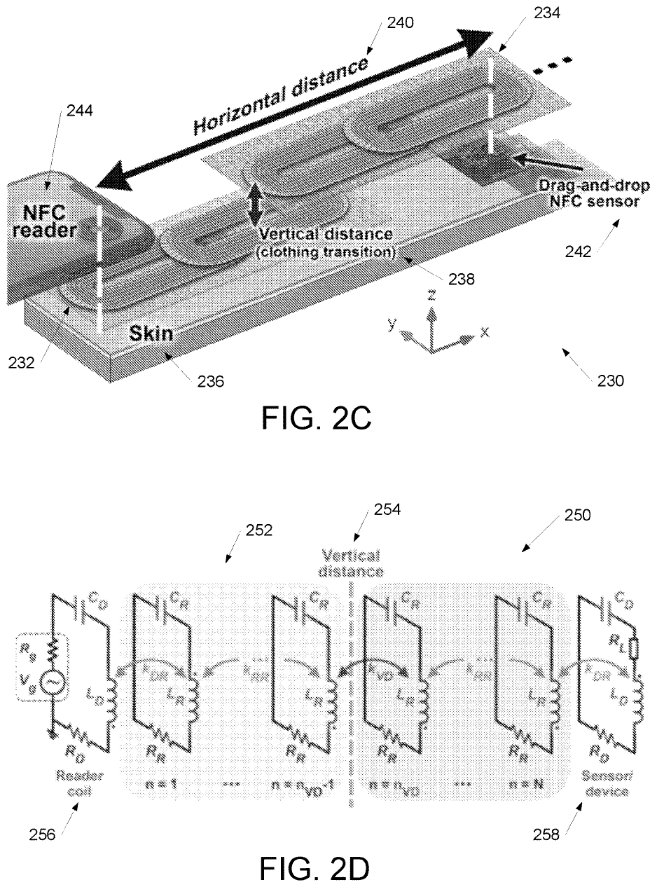

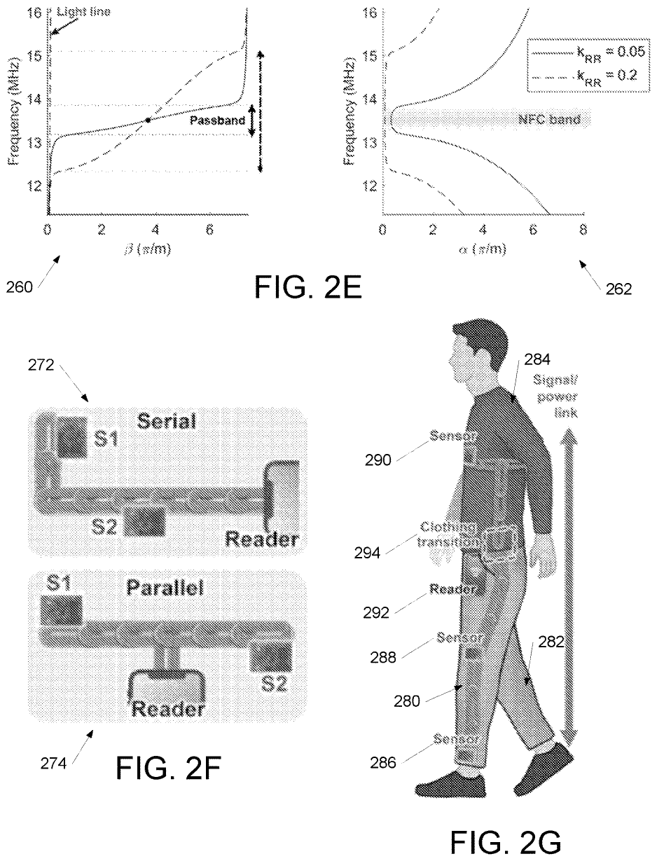

A schematic diagram illustrating a planar magnet resonator in accordance with an embodiment of the invention is shown in FIG. 2a. The planar magnet resonator 202 may include a flexible planar coil 204 and a ground layer 208. In some embodiments, the flexible planar coil 204 and/or the ground layer 208 may be on vinyl 206. The planar magnet resonator 202 may be placed on top of clothing 210 that may be on a person's skin 212. The distance 214 between the skin and the resonator 202 is shown. Graphs illustrating a slotted ground layer minimizing spectral uncertainty due to a human body's parasitic effect in accordance with an embodiment of the invention is shown in FIG. 2b. Specifically, graph 220 is without ground layer and graph 222 is with a ground layer. In many embodiments, this may compensate for the power dissipation generated from the flow of image currents on the ground layer. Therefore, the slotted ground layer may intervene in between the loop and skin, eliminate the unpredicted spectral shift of the resonator, and help miniaturizing the loop while not significantly affecting the resonator's quality factor. The magnetic field in this scenario may still be allowed to flow below and above the resonator despite this ground layer.

The MI waves can propagate through more convoluted pathways involving arrays of magnetically coupled resonators. A schematic diagram illustrating an array of magnetically coupled resonators with a horizontal distance (in x direction) and a vertical distance in other directions in accordance with an embodiment of the invention is shown in FIG. 2c. Diagram 230 includes magnetically coupled resonators (may also be referred to as “coils”) 232, 234, on skin 236, having a horizontal distance 240 and a vertical distance 238. The NFC sensor(s) 242 may be dragged and dropped across the magnetically coupled resonators 232, 234. In various embodiments, this magnetic connection allows for more flexibility in terms of the resonators' 232, 234 relative placements and introduces a horizontal distance 240 within a network between the NFC reader 244 and sensor nodes (in x direction), in addition to the vertical distances 238 (VD) between two neighbor nodes (resonator/device) on different pieces of clothing (or z axis). Such networks show metamaterial behavior along the coils (x direction) and typical nearfield properties in other directions. Thus, the nodes (including reader and multiple sensors) in the close vicinity of the coil network would be electrically and/or magnetically connected. The network's equivalent circuit 250 comprised of N coupled coils 252 plus one reader 256 and sensor 258 with a vertical distance 254 in between is shown in FIG. 2d.

The present embodiments may assume that the current flowing in the n^{th} resonator has a sinusoidal time-dependency with an angular frequency of ω. Here, the resonator-coils each with an impedance of

$${Z}_{R} = {R}_{R} + j ω {L}_{R} + \frac{1}{j ω {C}_{R}} ,$$

may be inductively coupled to their closest neighbor resonator with the mutual coupling of M_{RR}=k_{RR}L_{R} where M, k, and ω represent the mutual inductance, coupling factor, and angular frequency respectively (index RR shows inter-resonator relations). For simplicity, it may be assumed the vertical distance is ignorable (k_{RR}=k_{VD}). Further, it may be assumed a sinusoidal waveform for the current running on the n^{th} resonator (ranging from 1 to N) in an array as:

$${I}_{n} = {I}_{1} {e}^{j {ϕ}_{1}} {e}^{- j γ ( n - 1 ) {d}_{c}}$$

where γ is the propagation constant, I₁ and φ₁ are the first loop's current magnitude and phase depending on the excitation (boundary conditions imposed by reader's V_{g}), and d_{c} is the distance between two neighbor coils. The Kirchhoff's voltage law for the n^{th} coil follows:

$${Z}_{R} {I}_{n} + j ω {M}_{RR} ( {I}_{n - 1} + {I}_{n + 1} ) = 0$$

which leads to the dispersion equation:

$$γ = \frac{1}{{d}_{c}} \times {cos}^{- 1} ( \frac{- {Z}_{R}}{2 j ω {M}_{RR}} )$$

To consider harmonics of the standing wave, we define γₘ=4γ/m where m (≥2) indicates the number of coils that induce a phase shift of 2π on the standing wave. Here, γₘ=β−jα is the harmonic propagation constant (β as the phase and a as attenuation constants), and is calculated for our typical resonator electrical properties and shown in FIG. 2e. Dispersion diagrams 260, 262 for an array of resonators for various magnetic coupling coefficients in accordance with an embodiment of the invention is illustrated in FIG. 2e. The lower and higher cutoff frequencies (MI wave passband) are marked by dashed lines. The propagation constant profile β(ω) may possess different values depending on the coil geometry (reflected in Z_{R}), coupling factor, and harmonic modes (m, which is not necessarily an integer).

As shown in FIG. 2e, the metamaterial's passband may be tuned by the inter-resonator coupling (k_{RR}) or equivalently the neighbor coil distancing (d_{c}). Unlike traditional BANs that utilize coils connected by wire, in the present embodiments, the inter-resonator magnetic coupling enables complex network architectures with user-friendly extensions such as inline or fork connections.

Diagrams illustrating a reader and multiple sensors utilized in an in-line serial 272 and a T-shaped parallel 274 array of resonators to form various signal paths around a body in accordance with an embodiment of the invention are shown in FIG. 2f. When integrated into clothing, this may allow for more complex local signal paths suitable for BAN across multiple layers of disconnected clothing (e.g. from pants to shirts), and warrants more suitable use over the conventional BANs relying on wire- or conductive thread-based connection.

A diagram illustrating a metamaterial network 280 streamlined into separate clothing pieces in accordance with an embodiment of the invention is shown in FIG. 2g. The metamaterial network 280 may be easily streamlined into separate clothing pieces (e.g., pants 282 and shirt 284 having a clothing transition 294), enabling high flexibility necessary for daily routines and significant horizontal range extension. The metamaterial network 280 may include various components such as, but not limited to, sensor(s) 286, 288, 290 and reader(s) 292. The nodes may be placed anywhere close to (within a few centimeters of) any point of the network.

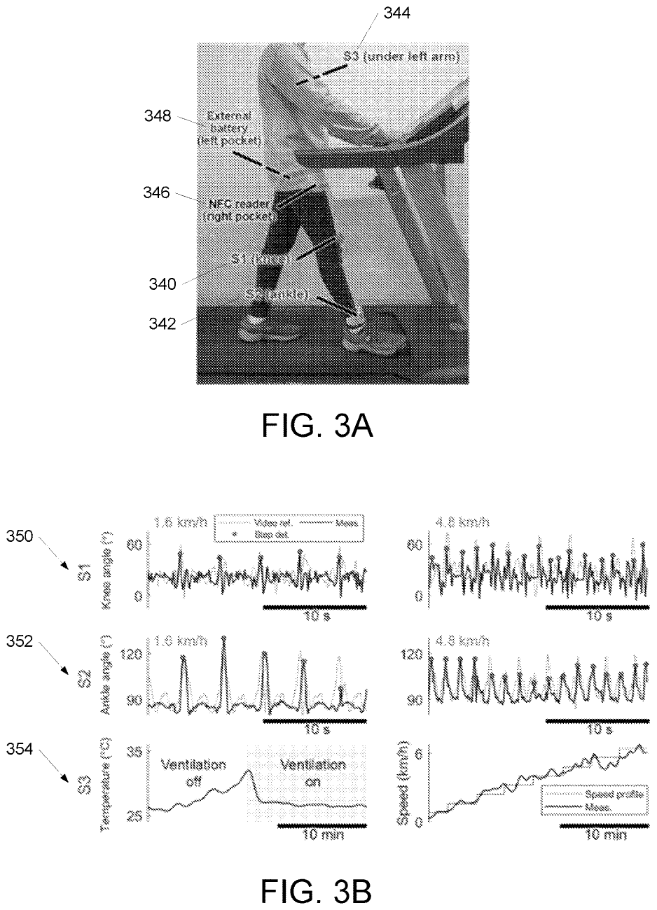

In reference to FIGS. 3a-b, network performance was evaluated by various readers and sensors, that just need to be placed close (e.g., 3 cm) to any point of the resonator chain. The initial sensor board may be based on a commercially available NFC transponder chip integrating an A/D unit that enables connection of a wide range of analog sensors (strain, temperature). In addition, seamless body-to-body communication is possible with no need for terminals. A link readily establishes by putting two networks in proximity, enabling person-to-person or clothing-to-clothing power transmission. Sensor networks were validated during strenuous daily activities.

FIG. 3a illustrates a high speed, long-term indoor walk/running activity measurement in accordance with certain embodiments of the invention. In many embodiments, test may be performed under a gradually increasing velocity profile for 25 min and BAN may be integrated into clothing with colored vinyl. In various embodiments, the BAN may include sensors 340, 342, 344 and NFC reader 346. In some embodiments, the BAN may also include an external battery 348. Diagrams 350, 352, 354, monitoring of sensors 340, 342, 344, respectively, during indoor running under various velocity profiles with a sampling rate of 10 Hz/sensor in accordance with an embodiment of the invention is shown in FIG. 3b. In various embodiments, steps may be detected and marked by circular markers.

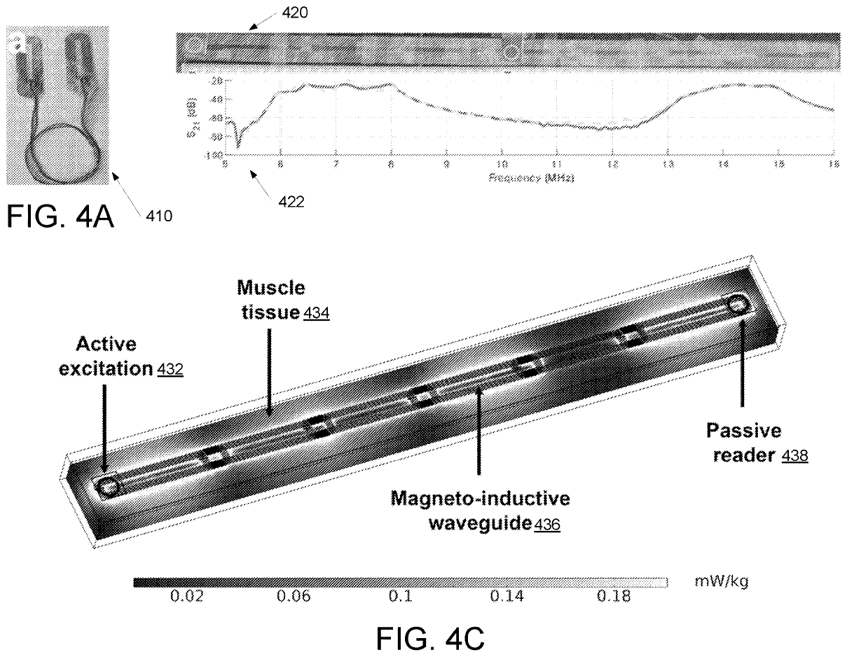

FIGS. 4a-c show preliminary validation for metamaterials exhibiting advanced control of MI waves. FIG. 4a is a diagram illustrating a jumper element 410 in accordance with certain embodiments of the invention. FIG. 4b illustrates an engineered multi-passband metamaterial element 420 and a graph 422 illustrating a multi-band network transmission profile for inter-coil (horizontal) coupling versus vertical coupling generated by a vertical distance in the middle of the network in accordance with certain embodiments of the invention. FIG. 4c illustrates a multi-passband metamaterial element 430 showing active excitation 432, muscle tissue 434, magneto-inductive waveguide 436, and a passive reader 438 in accordance with certain embodiments of the invention. FIG. 4c also show that SAR at 1 W at transmitter exhibits 2 orders of magnitude lower SAR than allowable limits in accordance with certain embodiments of the invention.

In reference to FIGS. 4a-c, two examples are highlighted here demonstrate spatial control of nearfield and transmitted bands. First, the present embodiments have demonstrated controlled regions of nearfield active/non-active by interceding an inductive element that may be referred to as a “jumper.” Here, two small coil elements are connected over long range by a low-loss connection. When interceded into the network as an element, wave propagation is still supported, however a null point appears as the wave effectively disappears and reappears across arbitrarily long points of the jumper. This element allows RF fields to spatially localize at desired points and improve transmission. A second structure supports engineered frequency bands. Resonant circuits may support multiple modes, however these appear at set frequencies as determined by resonator geometry and are uncontrolled. The present embodiments have developed multiple strategies that enable the propagation of multiple engineered frequencies. By interceding a tuning capacitor in elements, the passband of the metamaterial can be engineered to allow propagation of multi-designer frequencies (e.g., 6.7 MHz/NFC). This may allow enhanced control of wireless power, stimulation, or information transfer. Further, the utilized metamaterials exhibit low bodily absorption (i.e., SAR) as RF magnetic fields are weakly absorbed by tissue.

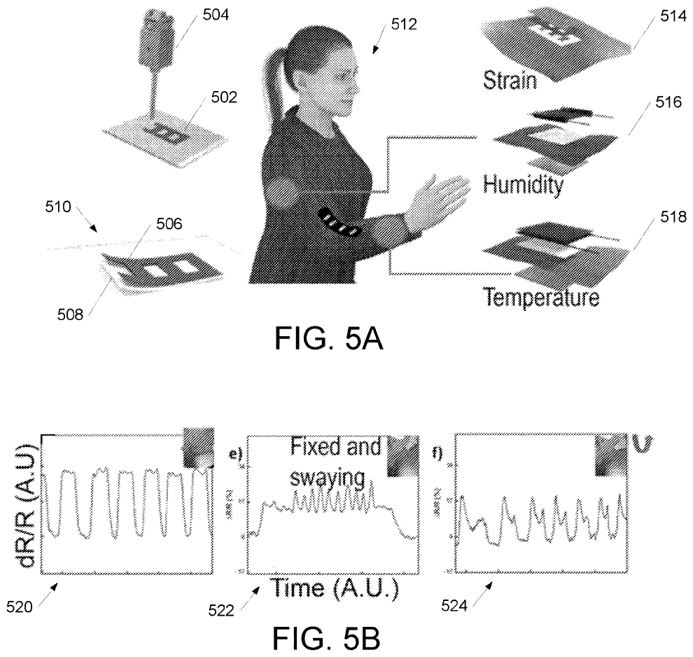

Textile-integrated, multifunctional sensors: In addition to textile-borne BANs, the present embodiments include sensors that integrate seamlessly on textile using similar low-cost integration steps. A diagram illustrating textile sensors based on laser-machined graphene in accordance with certain embodiments of the invention is shown in FIG. 5a. The platform is based around laser-induced graphene (LIG) 502, an emerging technique of generating low-cost, conductive carbon electrodes or sensing elements. Here, high power laser 504 is used to transform/pyrolyze polymers into nanostructured conductors. These can then be affixed stably on textile 508. These create versatile sensors 510 and the present embodiments have demonstrated on-body 512, textile sensing of strain 514, temperature 518, sweat and humidity 516. In some embodiments, the sensors may also include various types of perspiration sensors such as, but not limited to, sweat pH sensors, perspiration rate sensors, ion-selective sensors, etc.

Graphs 520, 522, 524 illustrating differential response of strain sensors to motion in accordance with certain embodiments of the invention are shown in FIG. 5b. Strain sensors were stable across 100000s of cycles and could measure unique motions when integrated into textiles along specific nodes.

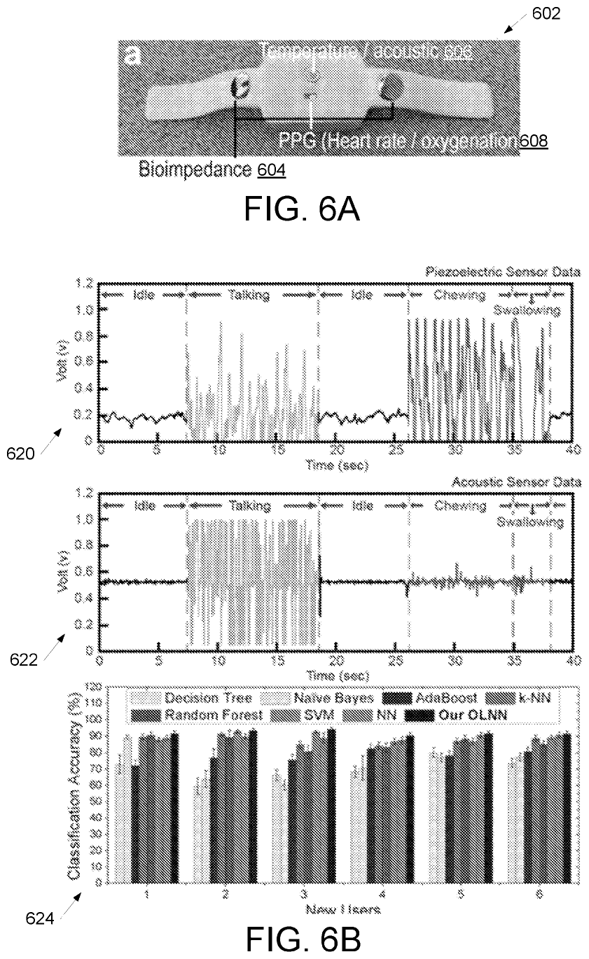

Wearable devices, PPG and machine learning: A diagram illustrating a multisensor wristband for neonates in accordance with certain embodiments of the invention is shown in FIG. 6a. In some embodiments, a neonate wristband 602 may be utilized for various conditions including, but not limited to Neonatal Abstinence Syndrome (NAS) monitoring. The neonate wristband 602 may include various sensors including, but not limited to, bioimpedance sensor(s) 604, temperature/acoustic sensor 606, PPG sensor 608, etc.

Graphs 620, 622, 624 illustrating machine learning (ML) on a multisensory device for intake monitoring in accordance with certain embodiments of the invention is shown in FIG. 6b. One example device is a multi-sensor (PPG, bioimpedance, acoustic, accelerometer) wristband for neonates. Further, wearable sensing nodes may utilize either NFC communication protocols or be powered wirelessly using AirFuel/Qi. Graph 620 provides data for a piezoelectric sensor. Graph 622 provides data for an acoustic sensor. The present embodiments may include edge-based, time-series data processing of embedded and cyber-physical systems (biological, automotive, and manufacturing). Efficient Edge AI frameworks such as binary convolutional networks and early exits may be used in conjunction with multi-sensors and modalities (including PPG) for event detection, which include withdrawal, circulation, eating and human activity, myocardial infarction, etc. Graph 624 provides classification accuracy for new users in accordance with certain embodiments of the invention.

Although specific data considerations for textile-BANs are discussed above with respect to FIGS. 2a-6b, any of a variety of data and data considerations as appropriate to the requirements of a specific application can be utilized in accordance with embodiments of the invention. Smart textiles and edge-AI considerations in accordance with embodiments of the invention are further described below.

### Smart Textiles and Edge-AI Considerations

The present embodiments include technical approaches along two lines: one focusing on “smart” textile design/optimization, and the other on embedded systems and edge-AI. Systems and descriptions focus on applications in CHD, however, the present embodiments are meant to scale, adapt, and generalize to many conditions that require full-body monitoring.

BAN Layout and Design: The BANs may be produced using techniques extended from vinyl t-shirt production, wherein discrete elements (e.g., sensors and magnetic resonators) can be designed, oriented, and affixed on clothing on-demand. This customizability is desirable; however, it can complicate characterization and optimization studies due to an overwhelming number of potential design choices. Thus, it is important to establish system layouts that can be used throughout including of the overall design of the network, and positioning of textile sensors. Establishing such layouts ensure that viewpoints from various fields (whether engineers, computer scientists, or medical practitioners) can be integrated.

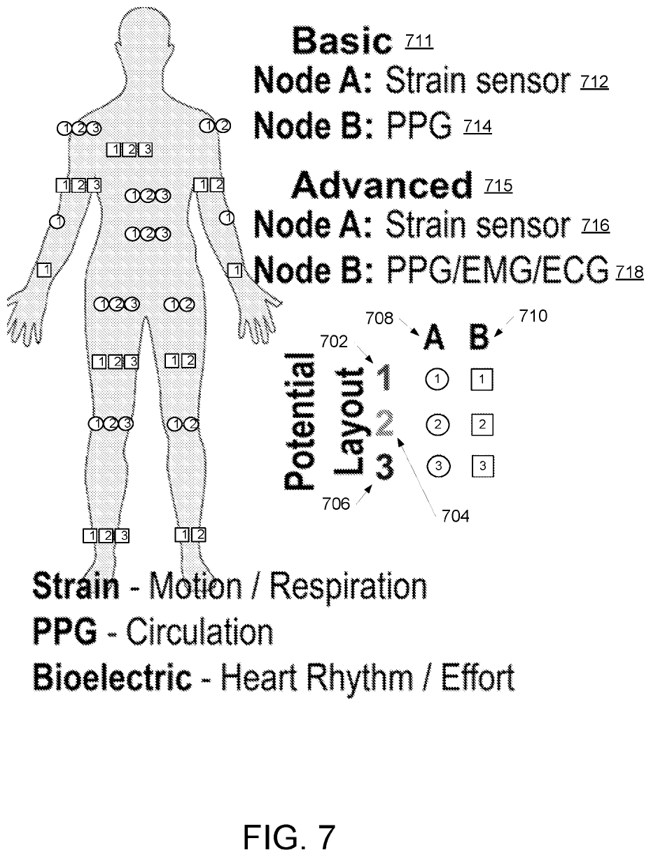

FIG. 7 is a diagram illustrating BAN layouts for CHD in accordance with certain embodiments of the invention. The initial three proposed metamaterial networks (i.e., potential layouts 1 702, 2 704, and 3 706 each layout with nodes A 708 and B 710) as illustrated may be formed on top of a basic long shirt/pants combination. Embedded into textile can be strain sensors (motion and respiration) and PPG monitors (circulation, pulse transit, blood pressure) that encompasses the basic BAN that may be tested in kids. For example, in a first set up (e.g., a basic configuration 711), the layouts 1 702, 2 704, or 3 706 may each include node A 708 utilizing strain sensors 712 and node B 710 utilizing PPG sensors 714. The more advanced BAN may additionally contain bioelectricity sensors for EMG and ECG. For example, in a second set up (e.g., an advance configuration 715), the layouts 1 702, 2 704, or 3 706 may each include node A 708 utilizing strain sensors 716 and node B 710 utilizing PPG, EMG, and/or ECG sensors 718. Potential sensor locations are shown in FIG. 7. As described above, the PPG can monitor heart rate anywhere, however SpO2 accuracy is more limited. Here, selected appendage locations have all been demonstrated in clinically validated devices for SpO2 monitoring. Exact sensor positioning may be decided and with knowledge of sensor distribution, the present embodiments properly test the limits and optimize network structure to improve performance. As described further above, in some embodiments, the sensors may also include various types of perspiration sensors such as, but not limited to, sweat pH sensors, perspiration rate sensors, ion-selective sensors, etc.

### Sensimetric “Smart” Textiles for Field-Adaptable, Wireless BANs

The present embodiments perform diverse engineering studies on advanced magnetic metamaterials for BANs and textile-integration of sensors and systems for CHD monitoring. Networks, sensors, and systems may be designed, optimized, fabricated, and validated on adults in controlled settings.

A first objective includes optimization of advanced magnetic metamaterials for resilient, adaptable wireless BANs. Magnetic metamaterials form the underlying network of the present wireless BANs. This approach simplifies interfaces (devices can be placed anywhere along to the pathway) and removes a key failure mode (wiring disconnections). In addition, these enable clothing-to-clothing signal transmission for full body monitoring.

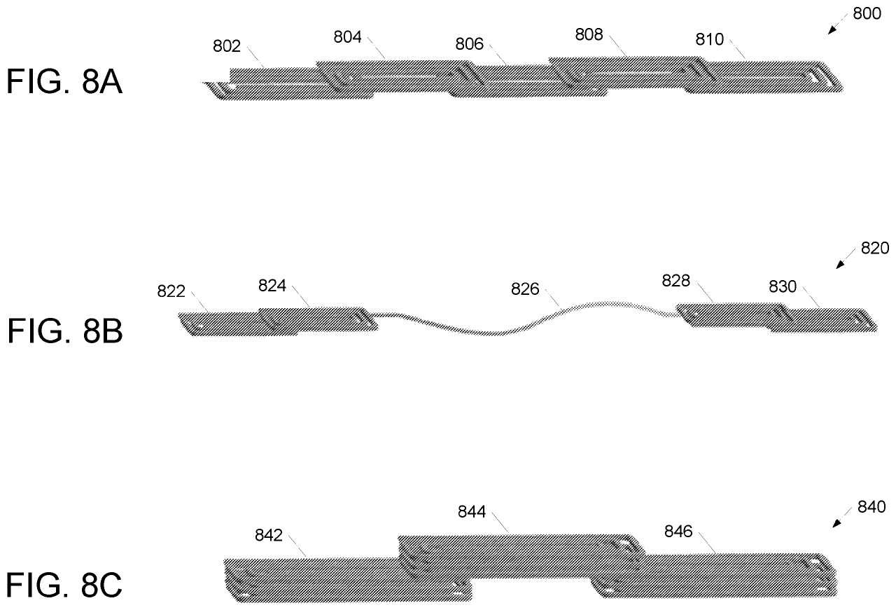

The optimization of advanced magnetic metamaterials may include the development of advanced magnetic materials for augmented control of on-body RF magnetic fields. Magnetoinductive waves traditionally propagate via a static, linear array of inductors that exhibit repetitive structural/spectral characteristics—these are used to transmit data of a single dominant frequency band. The present embodiments include novel strategies that will introduce more programmability/versatility into magnetic metamaterial networks for BAN applications. Diagrams illustrating advanced magnetic metamaterials for augmented BANs in accordance with certain embodiments of the invention is shown in FIGS. 8a-c. FIG. 8a illustrates a multiband near-field waveguide 800 that may allow propagation of multi-designer frequencies (e.g., 6.7 MHz, 13.56 MHz, etc.). The multiband near-field waveguide 800 may include a chain of coils of various lengths. For example, the waveguide 800 may include a first coil 802 is connected to a second coil 804 that is connected to a third coil 806 that is connected to a fourth coil 808 that is connected to a fifth coil 810. FIG. 8b illustrates a jumpered metamaterial waveguide 820 for improved transmissivity at on-off regions. For example, the waveguide 820 may include a first coil 822 connected to a second coil 824 that is connected to a third coil 828 via a jumper 826 for improved transmissivity. In some embodiments the third coil 828 may be connected to a fourth coil 830. FIG. 8c illustrates a stacked conductor 840 for improved transmissivity. The stacked conductor 840 may include a first stack 842 of coils connected to a second stack 844 of coils that is connected to a third stack 846 of coils.

The present embodiments include the analyzing magnetic metamaterials that support the propagation of multiple designer frequency bands, including NFC (13.56 MHz) for secure, low-power sensors, and AirFuel (6.7 MHz) that can support higher power transmission. The present BANs utilize a mix of NFC sensor-systems as well as PPG/bioelectricity sensors that may require higher refresh rate than is possible with typical NFC readers. Beyond PPG, higher wireless power can potentially be used to perform more computation, enable more sensors, or enable actuators. By separating NFC and AirFuel transmission bands, the present embodiments ensure that higher-power requiring devices can be controlled and not interact with NFC devices. Here we focus on optimizing structures shown in FIG. 4c, where an interceding capacitor can tune the resonant frequency of the second mode of the inductor. Arrays of these structures support the propagation of MI waves at two designed frequencies. Designs of varying winding count/tunings may be built into metamaterial arrays and characterized for S21 parameters. In addition, the present embodiments include analyzing how inductor anisotropy may impact the transmission characteristics of these metamaterials.

Further, the present embodiments include analyzing approaches to improve the power loss over the structure. Due to the smaller sizes of the intended users (i.e., children), metamaterials may not extend over a long range, however improved transmission may reduce power consumption (lessening reader battery size) and enable more robust measurement throughout the day. The first approach is to improve the conductance of the structure itself: this can be done through stacking additional foil layers. Stacks of 2, 3, and 4 metal layers may be able to improve network S21 characteristics, while changes to flexibility can be measured. Further, the present embodiments include analyzing how a heterogeneous metamaterial structure can optimize the spatial properties of the network by creating active and null regions of near-field. This allows the network to retain adaptability/flexibility, while certain regions that will never support devices can become null points and improve network transmissivity. This can be accomplished by interceding a “jumper” element into the network. Various winding designs (diameter and turn number) can be analyzed for impact on the spectral properties of the network. Because this element is not structurally the same as the rest of the network, its optimal coupling coefficient within the network will vary with its structural properties.

The optimization of advanced magnetic metamaterials may also include engineering power delivery to sensing nodes along multi-band magnetic metamaterials. The present embodiments also provide functional considerations in power delivery that come with networks. In practice, actual delivered power will vary from S21 measurement (that occurs on tabletop with a VNA).

In some embodiments, a mobile component that can deliver wireless power for the present BANs may be considered. This may only be required for AirFuel, as NFC has well-established readers. This design is based on a full bridge converter that converts a DC power source into an AC signal with 4 transistors in the power loop. A design advantage is that the power output can be easily controlled by increasing or decreasing the supplied voltage. RV1S9060ACCSP optocouplers may be used to isolate the power section of the device from the microcontroller, this integrated circuit (IC) is ideal due to its operating voltage and high-speed switching. GaNFets may be controlled by a TMS320F28022 microcontroller. With higher power transmission possible in the AirFuel band (around 1 W), NFC devices may require some additional shielding. If necessary, this can be implemented with high-pass filters connected next to the NFC antenna.

In shorter networks a standing wave pattern forms along the metamaterial leading to regions of low and high power transmission. Several approaches can be undertaken to minimize this. Frequency hopping by the transmitter can flip the standing wave nodes, ensuring all devices are exposed to enhanced transmission. A simpler approach is to terminate the network to minimize reflected wave intensity. Given limited space in metamaterial networks implemented on children, the most basic termination is to simply load end-metamaterial elements with resistors. Strategies may be compared for the practical, average delivered power from the wearable transmitter to receiver nodes on simple linear metamaterial networks.

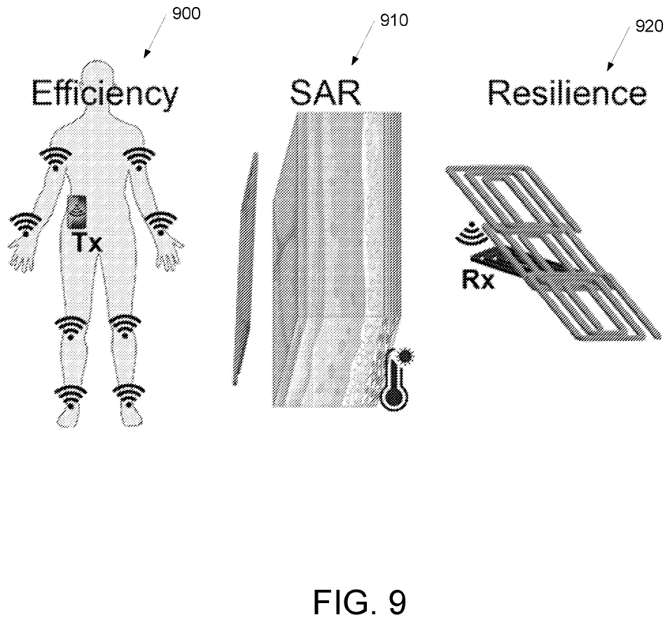

In addition, the optimization of advanced magnetic metamaterials may also include establishing FOM of advanced metamaterials in BANs. FIG. 9 is a diagram illustrating FOM in accordance with certain embodiments of the invention. One FOM is the delivered power (e.g., efficiency 900) given layout. Networks can be built on shirts/pants combinations (targeting children of middle school age and medium in size) according to designs optimized as further described above. The underlying substructure of the metamaterial may comprise 2 variations, including a basic (single metal foil, no jumper elements), and high transmission (multi-layer metal foils, jumper elements) version. In some embodiments, the S21 of the network may be measured using variations in input and output position. The primary reader (or transmitter) is typically located at the midpoint of the body (this optimizes transmission), and thus should allow for best performance with this arrangement. Note that inductive elements can readily be resized/tuned to precisely match any shirt design. From our studies, it is expected that ~1 W input power will be able to deliver at least ~20 mW to ~100 mW to extremities depending on basic or high-transmission substructures. This is more than sufficient for PPG/EMG measurement. No issues are expected with NFC, as working NFC BANs have been demonstrated in adults.

In reference to FIG. 9, the SAR 910 of these networks may be simulated using abbreviated network designs in CST microwave studio. Full network simulation is typically unnecessary because the maximum SAR occurs at the head of the network, this ultimately is what will limit the transponder power. SAR near the head of the network may be tested given basic or more transmissive metamaterial substructures, with input power from 1 W to 100 W. It is not expected to delivery such high power, however it is interesting to look at practical limits in power delivery. From results, it is expected that SAR will be minimal due to the weak absorption of RF magnetic fields in tissue. Combining S21 and SAR results, the present embodiments can identify fundamental limits in terms of power delivery (SAR limit is 1.6 W/kg).

In further reference to FIG. 9, the present embodiments also seek to understand the mechanical resilience 920 of textile magnetic metamaterial networks. For example, understanding how misalignment between the receiver node and the network may impact the delivered power. This may occur due to misalignment during clothing production (where the NFC antenna is improperly aligned to metamaterial), or more generally when the metamaterial deforms away from an on-body node. In some embodiments, the networks have no on-body nodes, however they are possible as further described below. Support of on-body or implantable nodes is additionally a key feature of the present networks. Thus, the present embodiments include studying x-y-z and angular misalignments and their impact on transmitted power. To characterize potential misalignments, simple studies may be undertaken with tight-fit and loose-fit clothing, wherein body motions are tested for their impact on positional misalignment. It is generally expected that tight-fit clothing will lead to significantly lower misalignment during motion than looser clothing. Encountered alignments may be tested on benchtop using a VNA, wherein a custom receiver coils may be varied in alignment with a linear metamaterial using a micromanipulator (matching tested misalignments on humans). S21 may subsequently be measured. In addition to practical variation in transmitted power during motion, the present embodiments characterize misalignment tolerance given power requirements, which may inform the type of clothing or any mechanical straps that may be required to minimize local textile deformation.

A second objective includes textile-integrated sensor-systems for advanced CHD monitoring. In many embodiments, directly embedded within textile are distributed, wireless-powered sensor-systems. These are automatically powered/networked via alignment with the magnetic metamaterial to perform cross-body measurements.

Textile-integrated sensor-systems for advanced CHD monitoring may be optimized with consideration of strain/bioelectric sensor design and optimization. Fused on textile may be strain sensors characterized above. These may be used to gather detailed data on specific exercises/body stressors and respiration. Sensor design may be optimized for target measurements (this may include length, turns, and exact positioning on textile). Such designs heat-pressed onto tight, fitted shirts/pants along joints, and sensor sensitivity characterized during targeted motion. Another important monitoring includes respiration monitoring, which may occur through a strain sensor embedded along the chest. Sensor designs may extend along the chest, and sensitivity compared during equivalent respiration events.

Bioelectricity can additionally be measured using laser-induced graphene sensors. Sensors can be further stabilized against strain through additional polymeric scaffolding, however in general strain modulation of electrode resistance does not significantly modify open circuit measurement of bioelectric signals. Similar textile graphene sensors are used in armbands to measure EMG. Here, EMG/ECG sensors may be implemented alongside PPG sensors in the same node, and the present embodiments implement strategies to ensure proper contact between this node and the skin.

Textile-integrated sensor-systems for advanced CHD monitoring may also be optimized with consideration of microsystems design and textile integration. In addition to elements that can be integrated into textiles using heat-press (e.g., laser-induced graphene sensors and magnetic metamaterials), printed circuit board (PCB) borne microsystems may need to be integrated into clothing to enable sensor readout. The present embodiments consider two separate microsystems. The first is NFC microsystems/transponders for strain sensor measurements. TI RF430 transponders may be integrated onto flexible PCB with an embedded antenna (tuned to 13.57 MHz). The second are Bluetooth-based microsystems that may be used to readout from PPG and bioelectric sensors, potentially using low-power Si117x PPG sensors (that contains a bioelectric frontend) and Nordic BLE microcontrollers. Systems may be built on flexible PCBs—the bottom layer houses the PPG, while the top layer contains the Nordic microchip, RF power converters, and an embedded antenna (tuned to 6.7 MHz).

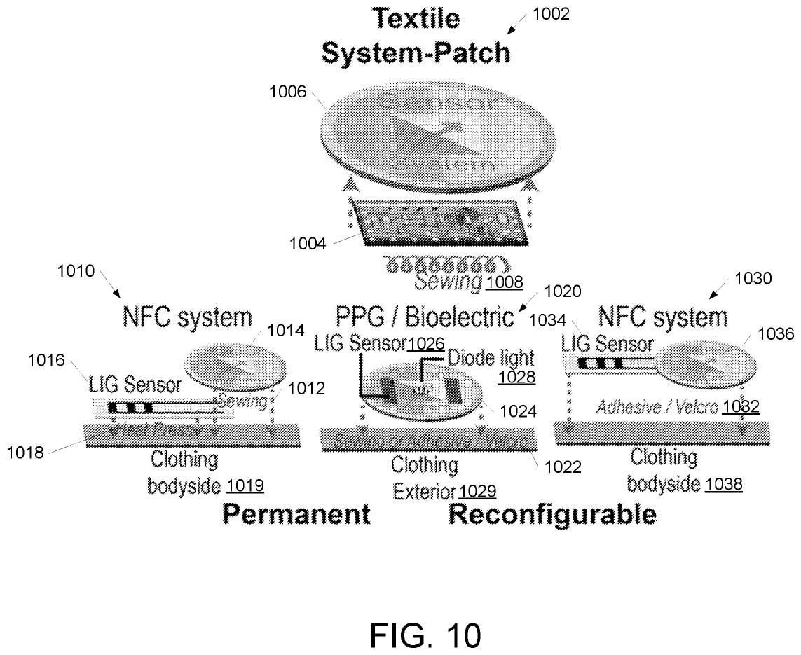

FIG. 10 is a diagram illustrating permanent and/or reconfigurable systems integration into textiles in accordance with certain embodiments of the invention. For example, textile integration of sensors and microsystems using a textile system-patch 1002 is illustrated. For example, a textile system-patch 1002 may be generated by attaching a flexible PCB 1004 to a textile 1006 (e.g., embroidered, twill). Systems textile integration may occur via permanent sewing 1008 or a modular, Velcro/adhesive approach enabling field-adaptability. This strategy is akin to modern approaches in skin-borne devices, namely to ensure that microchips are encapsulated, sealed, and integrated with soft or flexible components. Despite flexibility, it is still desirable that these microsystems are somewhat stiffer than the textile itself—here systems may be integrated into embroidered patches or badges that are common on clothing.

In reference to FIG. 10, NFC-system integration may occur on the exposed clothing side, while PPG may occur on the interior clothing. For example, NFC system integration process 1010 may include heat pressing (1018) various sensors (e.g., a LIG sensor 1016) to an exposed clothing side (opposite the clothing bodyside 1019) and sewing (1012) a textile 1014 on to the exposed clothing side. In another example, an NFC integration process 1030 may include attaching 1032, utilizing an adhesive (e.g. Velcro), various sensors (e.g., an LIG sensor 1034) and a textile 1036 to an exposed clothing side (opposite the clothing bodyside 1038). In a further example, a PPG/bioelectric system integration process 1020 may include attaching (1022) by either sewing or utilizing an adhesive (e.g., Velcro) a textile 1024 to a clothing bodyside (opposite a clothing exterior 1029). In many embodiments, the textile 1024 may include various sensors including, but not limited to, a LIG sensor 1026, diode light 1028, etc. Typically, PPG/ECG/EMG must touch the skin (ideally with additional seal pressure) and typically require additional stabilization. Strain sensors may be extended laterally to accommodate electrical connection with the flexible PCB. Wiring may occur with a thin wire with several bends to improve flexibility, and system interface first sealed/affixed with fabric glue, and the entire microsystem encapsulated with spray-on silicone. For the fixed network, the PCB may be stitched into clothing (e.g., through designs), and a final textile patch covering the layer stitched to form the final structure. The PPG may similarly occur on the inside textile. It may be first affixed with fabric glue, encapsulated with spray-on silicone, sewn into the clothing, and partially sealed with a dark fabric cover. An alternative strategy using Velcro or fabric tape may be used to create adaptive networks. Several strategies may be implemented to ensure flush connection of the PPG (optionally EMG/ECG) to skin.

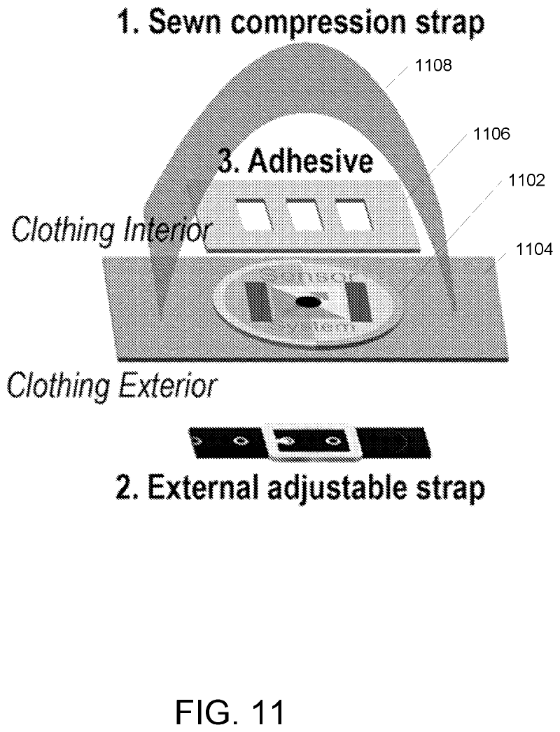

FIG. 11 is a diagram illustrating textile stabilization techniques in accordance with certain embodiments of the invention. The process may include sewing a band 1102 directly into the interior 1104 of the garment, applying adhesive tape 1106, and wrapping a band 1108 around clothing after initial dress.

Textile-integrated sensor-systems for advanced CHD monitoring may also be optimized with consideration of performance of textile-integrated sensor-systems. While sensors and systems have previously been validated, the full textile-integrated system has not. Here, the present embodiments include a variety of tests on the present textile systems. A first study relates textile-integrated PPG/EMG/ECG to compare strategies to ensure flush connection. A spaced array of 3 nodes (powered by button batteries) may be aligned along the arm using 3 techniques (e.g., internal cloth band, adhesive, and exterior band), and measurements during exercises compared for noise, repeatability, and stability. It is expected that the combinations of adhesive and bands to perform the best, however ideally less stabilization may be needed.

In addition, the present embodiments include analyzing the networked textile sensor-systems. For example, 2 abbreviated networks built on fitted/compression long shirt/pants may be analyzed. One along the arm (2 NFC, 2 BT), and second along the leg (2 NFC, 2 BT). A first characterization may occur using repetitive stretching exercises (with and without weights), validating textiles exhibit readout under high deformation. Next characterization may be over a 30-minute run/walk. In addition, the present embodiments include ensuring that the present permanent systems can survive washes (metamaterial networks themselves are robust to fairly aggressive washes). In some embodiments, microsystems may be sealed with spray-on silicone and may be water-proof. Further survivability can be achieved by temporarily applying additional fabric tape (or silicone) over microsystems to seal specific areas or prevent deformation during washes). Various strategies may be tested for effect on textiles in mild to aggressive wash steps.

In regards to textile-integrated sensor-systems for advanced CHD monitoring, full-body motion measurements can identify specific stressors (whether running, lifting, etc.) and their impact on circulation. However, other measurements also be useful and utilized with the present embodiments, including, but not limited to, temperature, sweat, humidity. Various measurements may be implemented using the LIG platform as described herein. In some embodiments, the AirFuel network may not deliver sufficient power to the PPG/EMG sensors. In the case that all wireless power delivery systems fail, button batteries may be attached to each PPG. This of course heavily increases burden and are non-trivial to charge but will ensure a working network. In some embodiments, PPG/ECG/respiration monitors placed near the chest may_not integrate properly with textile or provide insufficient measurement. While chest strap sensors are common, it is awkward to integrate these into clothing. In poor contact or integration scenarios, on-body, epidermal nodes may be used.

### Edge-AI for CHD and Full-Body Networks

A first objective of edge-AI for CHD includes edge-intelligence implementation and design. Optimization of edge-intelligence implementation may include consideration of wireless reader development (network head). In many embodiments, the reader processing head may be a custom embedded system running a silicon lab Gecko/Nordic MCU with Bluetooth Low Energy (BLE) support. It may wirelessly collect data from NFC sensors via the NFC transponder and handle Bluetooth data through the BLE advertisement protocol. It may also act as the central node for CHD abnormality detection. For ML data collection, data may be stored with timestamps to a local relational database such as SQLite. To handle time synchronization between NFC and BLE sensors readings, when the system is first activated, a delta time (Δ), which is Round-trip-time (RTT), is calculated for all the NFC and BLE sensors. The delta time (Δ) is stored in a database with sensor's identification such as MAC-address. Third, the actual sensor value is obtained through the BLE scanning process and NFC readout. The timestamp is calculated by subtracting delta time (Δ) and sensor data is stored with the timestamp. Depending on the sensor node type, preprocessing, and filtering may be done either locally at the compute capable nodes or at the reader processing head. All processing and ML code may be converted to C code, then converted to executable machine code on the respective target MCU.

To conserve energy, the system may be primarily in intermittent operation where NFC strain sensors may be sampled to detect stressors (running/jumping) and aberrant heart rate measured using a subset of PPG. This can be achieved by pushing very low energy into the AirFuel (50 mW), so that only 1 or 2 close nodes are activated. If either of these conditions are satisfied, the system may transition to active monitoring by turning on all sensors and the CHD ML pipeline. The network may remain in active monitoring until the prior two conditions are resolved. The abbreviated low power network may activate based on preset parameters (i.e., x second probes every y second). Specific settings may be investigated based on the tradeoff between energy savings and information gain/accuracy of event detection whereby increasing probe duration/frequency is expected to increase accuracy and response time of the system at the cost of higher energy consumption and lower system uptime.

Optimization of edge-intelligence implementation may include extraction of major parameters and activity recognition. The present embodiments may infer simple activities based on the amplitude/frequency of strain values and their corresponding detection locations using simple heuristics to determine hand movement, walking/running/jumping, breathing rate, etc. These activities (potentially combined with EMG) may be used for recognizing CHD stressor events and activating the full body monitoring. On the other hand, beat-to-beat analysis of multisite PPG pulses may be used to calculate PPG biometrics. These include pulse transit time (PTT), pulse wave velocity (PWV), wave morphologies such as amplitude, width, area, along with high level abstract time domain features such as SpO2, heart rate (HR), heart rate variability (HRV), blood pressure (BP), and frequency domain features such as discrete wavelet transforms (DWT), and short-time Fourier transforms (STFT). Together these form the inputs to the ML pipeline for CHD abnormality detection. Note, that activity recognition on its own may be fed into ML for improved stressor/event detection, and to identify specific actions and their characteristics. This becomes robust by increasing the number of sensors that are used to detect activities (EMG, accelerometers, etc.). However, in some embodiments, the primarily focus may be on abnormality detection within circulation.

A second objective of edge-AI for CHD includes ML, activity recognition, and intervention direction. Optimization of ML, activity recognition, and intervention direction may include consideration of domain invariant feature extraction and adaptation. The primary task of CHD monitoring is to detect abnormal heart conditions, however due to variations in patient biometrics and their unique cardiovascular characteristics that define what is normal/abnormal, standard ML may fail to generalize under these conditions. To address the domain shift problem, the present embodiment may use variational autoencoders (VAE) to learn domain invariant features. Given input vector x in [symbol] ⁿ, the latent feature z in [symbol] ^{d} may be inferred through an encoder that learns the conditional distribution P(z|x) by minimizing the Kullback-Leibler (KL) divergence against the true prior distribution P(z). Normally, P(z) is assumed to be Gaussian and captures a single latent space, but by utilizing representation disentanglement following [DIVA] the present embodiments can learn distilled features by “filtering” them into domain-specific (zd), class-specific/domain-invariant (zy), and input-specific (zx) latent features. Disentanglement is achieved by using domain identifiers like patient id (d), class label (y), and input (x) to estimate their respective subspace priors. Together with a decoder that learns P(x|z_{d}, z_{y}, zₓ) by minimizing the input reconstruction error, the present embodiments can learn salient features that accurately describe the given input. The autoencoder may include standard convolutional neural network (CNN) layers like batch normalization, nonlinear activations, etc. and classification may be performed using gated recurrent units (GRU) to capture the temporal correlations in the learned features. The autoencoder parameters are optimized by minimizing the following loss function:

$$L ( d , x , y ) = {𝔼}_{{q}_{{ϕ}_{d}} ( {z}_{d} \vert x ) . {q}_{{ϕ}_{x}} ( {z}_{x} \vert x ) , {q}_{{ϕ}_{y}} ( {z}_{y} \vert x )} [ log {p}_{θ} ( x \vert {z}_{d} , {z}_{x} , {z}_{y} ) ] - BKL ( {q}_{{ϕ}_{d}} ( {z}_{d} \vert x ) ‖ {p}_{θ} ( {z}_{d} \vert d ) ) - BKL ( {q}_{{ϕ}_{x}} ( {z}_{x} \vert x ) ‖ p ( {z}_{x} ) - BKL ( {q}_{{ϕ}_{y}} ( {z}_{y} \vert x ) ‖ {p}_{θ} ( {z}_{y} \vert y ) )$$

where the first term estimates the reconstruction likelihood, and the latter three terms estimate the KL divergence between the encoders and their respective priors (p_{θ} are learned by network parameters θ whereas p alone assumes Gaussian). Additional losses can be used to encourage information separation.

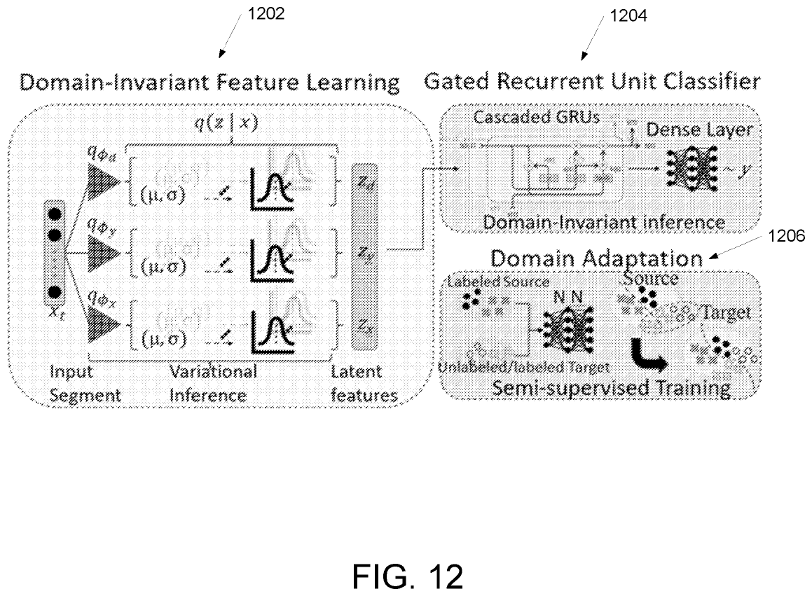

FIG. 12 is a diagram illustrating a domain agnostic framework in accordance with certain embodiments of the invention. An example of domain-invariant feature learning 1202 is provided. Further, an example of a gated recurrent unit classifier 1204 and domain adaption 1206 are provided. Although domain invariant features can minimize covariate shifts, i.e., subject input variances, by mapping features to the same latent space, still when faced with concept drift, unique heart rhythms causing deviations in the meaning or posterior distribution P(Y|X) of normal/abnormal cardiovascular functions, domain invariant features may not be sufficient, therefore domain adaptation techniques may also be applied to adjust the model to the new concepts or bias. This can be done by collecting target patient data as an initial calibration phase and used for retraining the model with the same loss function given labeled target samples at the expense of manual labeling or extending the loss function for semi-supervised learning given unlabeled data but may encounter training destabilization due to the quality of the unlabeled data. Training destabilization can be overcome through contrastive learning techniques, where only representative target samples, ones with the highest model uncertain, may be used to train the model. To evaluate the performance of the model, the present embodiments includes testing it on public datasets such as the Opportunity human activity dataset and the PTB/PTB-XL myocardial infarction detection dataset. The present embodiment may use the leave-one-subject-out validation to mimic subject induced domain shifts, additionally, sensor induced shifts can be experimented by swapping out sensors of the same type or leads.

Lastly, simple local intervention can be displayed to the user via the reader processing head based on the current activity and biometrics. For example, it can inform the person to stop running/climbing stairs and rest or in the presence of abnormal biometrics to suggest seeking doctor intervention or forward information to the user's primary care physician.

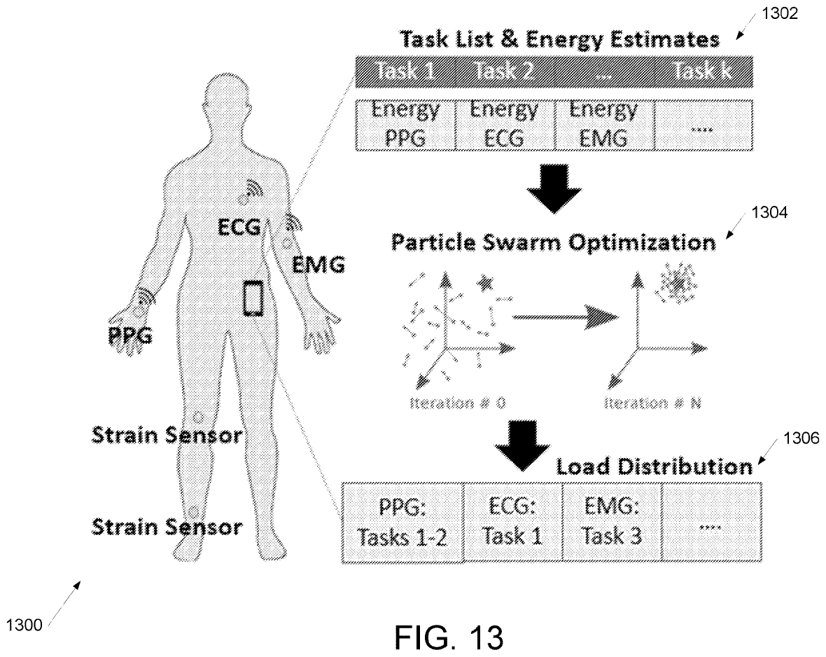

Optimization of ML, activity recognition, and intervention direction may BAN network optimization. With the potential addition of compute-capable battery-powered sensor nodes that may be on the body or on the textile, disparities in energy requirements for these sensor nodes can bottleneck system uptime and undermine overall system utility. To optimize such a network, the present embodiments balance the processing load by distributing tasks based on each node's current available energy. To do so, the present embodiments may utilize the particle swarm optimization (PSO) algorithm and define the following fitness function:

$$f ( T ) = {\sum }_{i = 1}^{N - 1} \vert {E}_{{node}_{i}} - {E}_{{node}_{i + 1}} \vert$$

$${E}_{{node}_{i}} = {E}_{{node}_{i}} - {E}_{local} - {E}_{Tx} - {E}_{Rx}$$

$${E}_{local} = \frac{{task}_{size}}{{node}_{f}} * {P}_{local}$$

$${E}_{Tx} = \frac{{task}_{size}}{{ϕ}_{Tx}} * {P}_{Tx}$$

$${E}_{Rx} = \frac{{task}_{size}}{{ϕ}_{Rx}} * {P}_{Rx}$$

where E_{node}_{ᵢ} is the estimated available node energy, E_{local} is the estimated local task execution energy given node frequency in bits/second, task size in bits, and E_{Tx}, E_{Rx} are the estimated transmit and receive energy respectively. Given N nodes and a set of tasks T=[t₁, t₂, . . . , tₖ], PSO will find the best distribution of tasks amongst the N nodes to minimize their energy discrepancies which should improve overall system utilization distributing tasks to idle nodes with abundant energy. The optimizer can be triggered in preset intervals to re-adjust the load distribution or be event triggered i.e., % drop in energy of any node over a given time frame. FIG. 13 is a diagram illustrating a PSO framework in accordance with certain embodiments of the invention. A PSO framework 1300 may include generating (1302) a task list and energy estimate, conducting (1304) particle swarm optimization, and conducting (1306) load distribution.

Given the difficulty and turnaround time for labeling target data in the case of domain adaptation, alternatively, test-time adaptation can be used to update the network on a per sample basis with online gradient updates in a continual online learning framework, but at the expense of additional runtime energy consumption. The present embodiments may also include validating both techniques based on their merits and challenges in the case of CHD through extensive empirical studies. On the other hand, the frequent invocation of the PSO optimizer may undo its energy gains, alternatively a simple static energy profile can be used based on runtime statistics of the sensor nodes for runtime system configuration.

Although smart textiles and edge-AI considerations are discussed above with respect to FIGS. 7-13, any of a variety of smart textiles and edge-AI considerations as appropriate to the requirements of a specific application can be utilized in accordance with embodiments of the invention. Functionalized textile-BANs for pediatric CHD in accordance with embodiments of the invention are further described below.

### Functional Textile-BANs for Pediatric CHD

The present embodiments also include testing the performance of basic (strain and PPG sensors only) field-adapted BANs targeting prepubescents. A first objective for optimizing functional textile-BANs for pediatric CHD includes consideration of performance of textile-BANs and edge-AI in adults. While technological elements such as the textile systems and edge-AI are validated as described above, several combined elements such as BAN on-off control, activity recognition, and ML training can be further validated. In many embodiments, the advanced BAN layout 2 (as illustrated in FIG. 7) may be built on fitted/compression t-shirts/pants for study in several healthy adults.

As this BAN is meant to be worn by users daily, the present embodiments may consider the behavior of the systems over many visits with variations on select activities (e.g., walking, isolated muscle training exercises). The on-off cycle of the BAN may be triggered by many potential conditions derived from the combination of PPG/motion measurement. Here, thresholds may be tested to ensure that the BANs toggle on during the initiation of exercises and consider how different conditions impact power utilization. In addition, the present embodiments consider accuracy of the heuristic models of activity, which should easily be able to detect events like walking, local muscle activity, strength utilization, and more.

In some embodiments, repeat data from subjects can be used to train the ML framework, as datasets do not typically exist for complex cross-body networks. Users may repeat activities in similar conditions/time of day to train the framework. In some embodiments, tests may determine whether either synthesized data, or data gathered in slightly more taxing scenarios (such as later in the day during fatigue, or after a lapse in workout) can be recognized by the AI.

A second objective for optimizing functional textile-BANs for pediatric CHD includes the validation of BANs in prepubescent children (ages 7-13). The focus here is to validate that textiles/BAN are properly utilized and working in this target group. The present embodiments may limit the age range to middle-to-late prepubescent children as this group is beginning to experience independence from constant adult supervision and has limited familiarity with technology. Thus, this group may be ideal to assess response to the modified clothing.

In some embodiments, the present embodiments may be validated on a sampling of heathy children (typically more active than children with CHD) to profile various facets of how children interact with, maintain, react, and stress-test the clothing. By testing on healthy children, the present embodiments are able to test a wide variety of strenuous activities in limited timelines. In addition, data from healthy kids may form the backbone of the ML framework. Youth sizes small (6-7), medium (8-9), large (10-12) fitted/compression/stretch t-shirt and pants may be structured with magnetic metamaterial networks (splitting elements will need to be sized/angled/tuned for respective sizes), sensors, and straps/adhesive on the chest, upper arm, leg, and ankle to ensure PPG to skin contact.

For example, 30 healthy children (ages 7 to 13) may each have 3 distinct visits of 2 hours. Visit 1 may be encompassed by a variety of short-term exercises in the gym (walks on the treadmill at varying pace; strength-training exercises targeting various muscles). Visit 2 may have free play where the kids can do as they please in an open area with objects to interact with. Visit 3 may have a mix of free play and exercises in a gym. BAN accuracy may be validated in gym activities (treadmill exercises and some basic gym work). To validate strain sensors, data may be compared with motion-tracking camera and/or accelerometer information. Circulation data may be compared with commercial fingertip/feet pulse-ox devices (Masimo, etc.). At various time points throughout, blood pressure extracted from multi-PPG may be compared using a medical-grade blood pressure cuff, alongside respiration tests.

From various structured/unstructured activities, the present embodiments assess how kids respond to the present textile systems and assess the power-control algorithms. In addition, this gathers a robust set of activity/bodily response data from which to train the ML network. Synthesized data from CHD patients exhibiting varying activity-circulation response profiles may be tested within the ML framework. There is limited existing data on such patients and the accuracy of the ML framework may depend heavily on data gathered from kids as they use the clothing.

Consideration is given to complications of full working textile. In some embodiments, given limitations in the integration of systems into the textile itself, or the failure of intermediate, wireless power delivery using metamaterials, a less technologically advanced BAN on kids may be considered. This may contain strain sensors distributed across textile magnetic metamaterials, in addition to on-body, battery-powered PPG nodes.

Although specific functionalized textile-BANs for pediatric CHD are discussed above, any of a variety of textile-BANs as appropriate to the requirements of a specific application can be utilized in accordance with embodiments of the invention. LIG textile considerations in accordance with embodiments of the invention are further described below.

### Laser-Induced Graphene (LIG) Textile Considerations

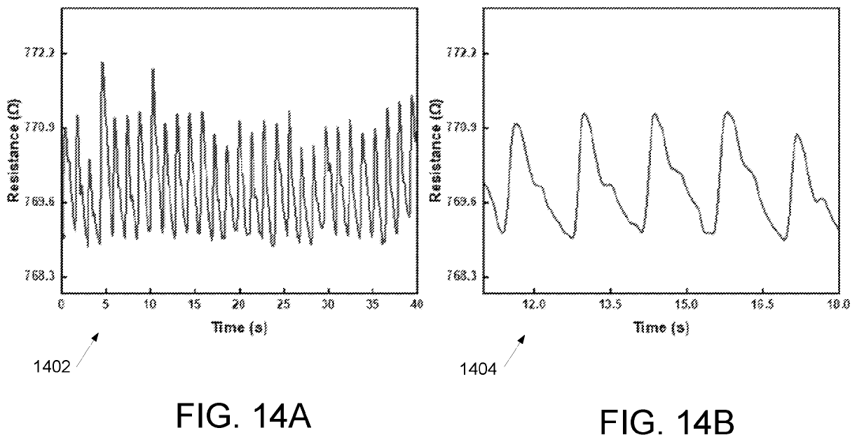

Graphs illustrating application of an LIG textile for pulse detecting in accordance with certain embodiments of the invention are shown in FIGS. 14a-b. Specifically, graph 1402 of FIG. 14a illustrates testing results of a wrist pulse signal detected by an LIG textile. Graph 1404 of FIG. 14b shows magnification of a few pulses of FIG. 14a.

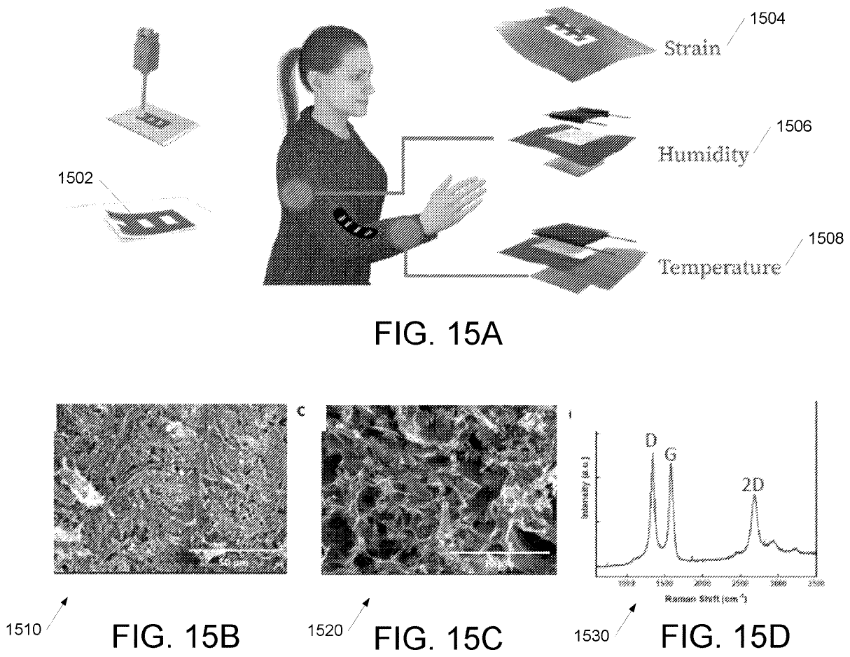

A graphene-based (e.g., LIG) multifunctional textile for integrated sensing platforms in accordance with certain embodiments of the invention is shown in FIGS. 15a-d. FIG. 15a illustrates the graphene-based multifunctional textile's 1502 ability for use as a strain sensor 1504, humidity sensor 1506, and temperature sensor 1508. Devices on different locations on the textile may monitor human motions, detect sweat humidity, and temperature change which can be further integrated with a wireless NFC readout platform (see also FIG. 5a and corresponding description above). FIGS. 15b-c show the morphology characterization of LIG under laser power of 9.5%. In FIG. 15b, image 1510 shows a 50 μm scale. In FIG. 15c, image 1520 shows a 10 μm scale. FIG. 15d provides a graph 1530 that shows the Raman spectra of LIG.

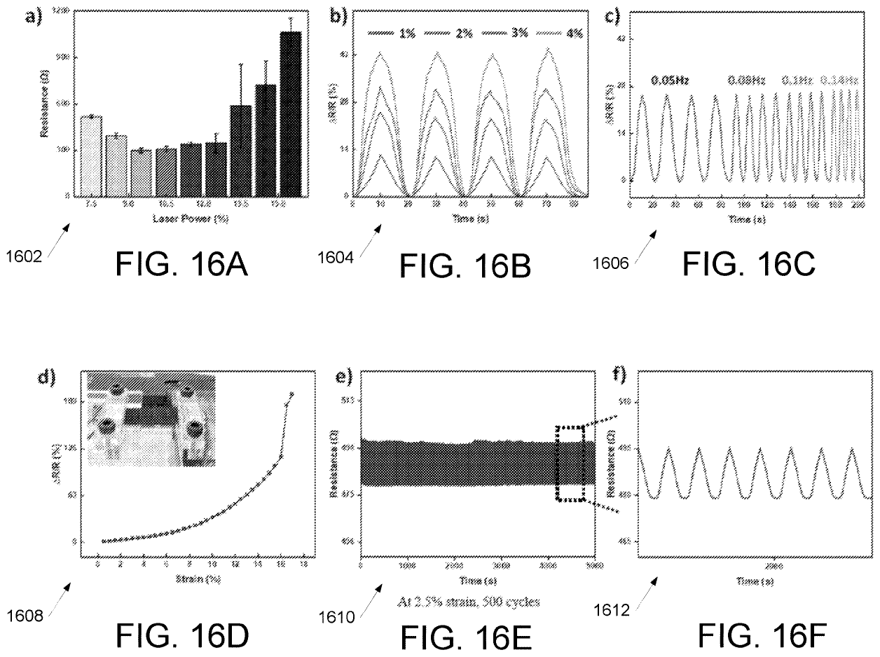

Mechanical performance of an LIG when used as a strain sensor in accordance with certain embodiments of the invention is shown in FIGS. 16a-f. FIG. 16a includes graph 1602 that illustrates a repeatability test of LIG under different laser power. FIG. 16b includes graph 1604 that shows the response of relative resistance at strain at 1%, 2%, 3% and 4%. FIG. 16c includes graph 1606 that shows the response of relative resistance toward 0.05, 0.08, 0.1 and 0.14 Hz frequencies. FIG. 16d includes graph 1608 that shows the relative resistance changes of the LIG under the different strain. FIG. 16e includes graph 1610 that shows lifetime testing of LIG at 2.5% strain for 500 cycles. FIG. 16f includes graph 1612 that shows a zoomed-in section of graph 1610 of FIG. 16e.

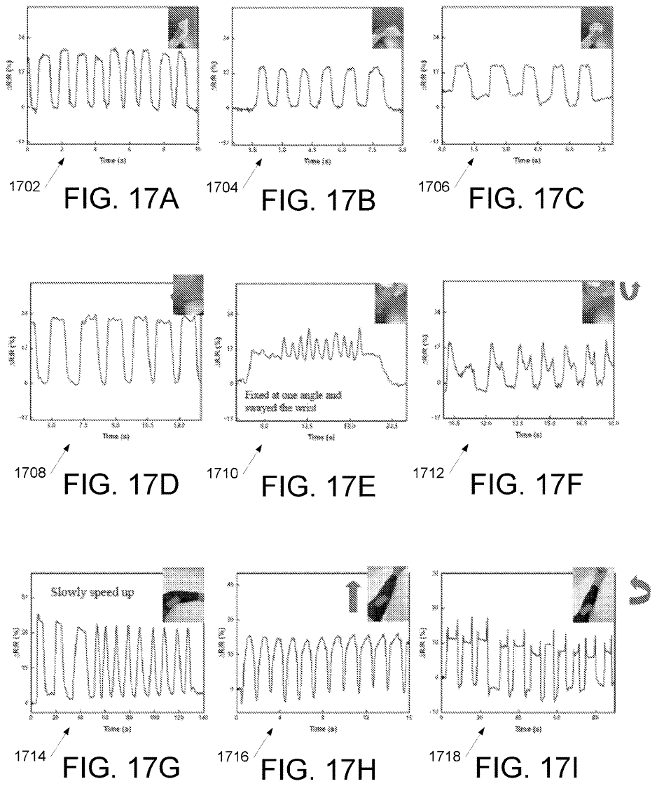

Detection of different human motions by a LIG used as a strain sensor in accordance with certain embodiments of the invention is shown in FIGS. 17a-i. FIGS. 17a-c includes graphs 1702, 1704, 1706, respectively that show strain signals of bending and turning of the wrist. FIGS. 17d-f includes graphs 1708, 1710, 1712, respectively that show strain signals of bending and turning of the elbow FIGS. 17g-i includes graphs 1714, 1716, 1718, respectively that show strain signals of bending and turning of the cubital fossa.

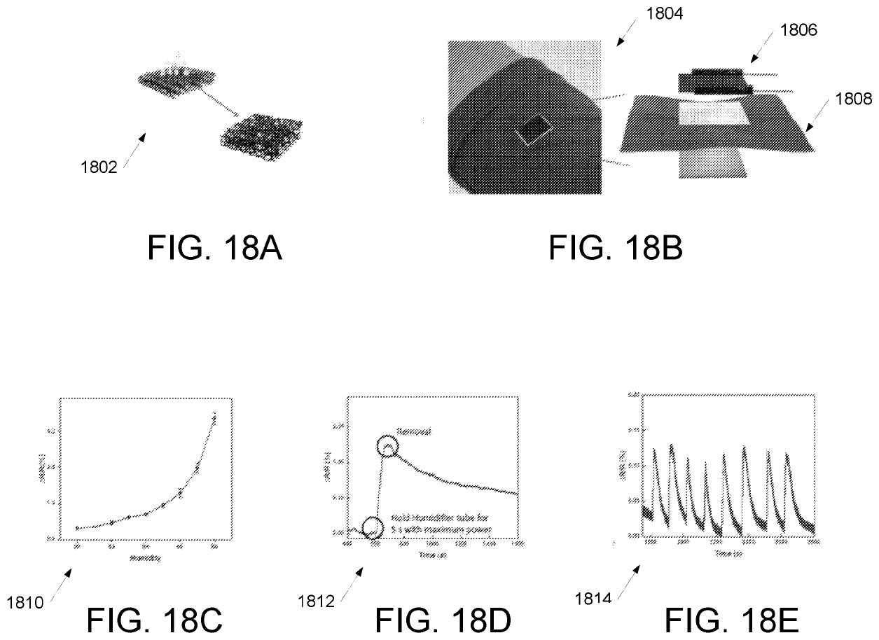

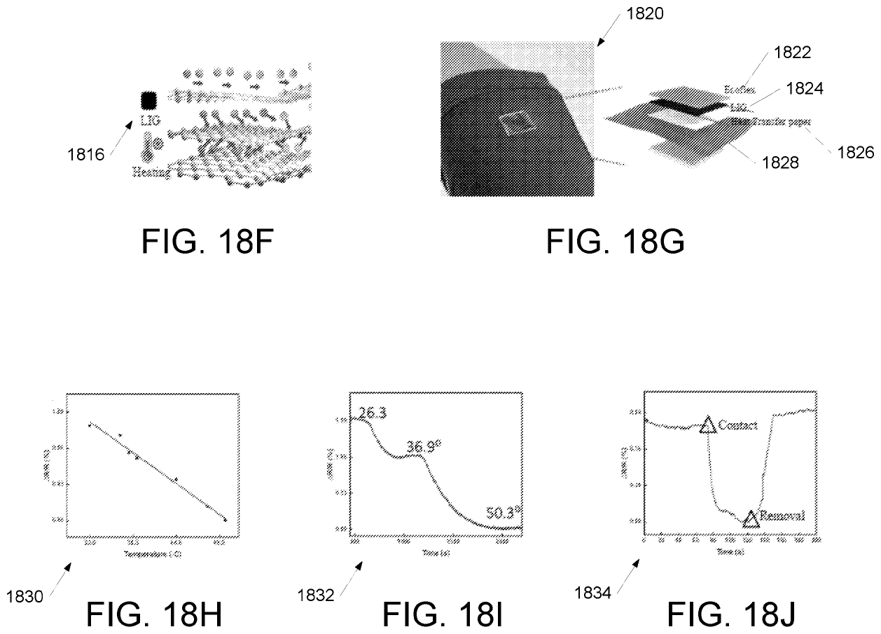

Design and characterization of an LIG-based humidity and temperature sensor in accordance with certain embodiments of the invention is shown in FIGS. 18a-j. FIG. 18a illustrates mechanisms 1802 of humidity sensing. FIG. 18b is a picture 1804 of the humidity sensor 1806 on the textile 1808. FIG. 18c includes graph 1810 that shows humidity characteristics. FIG. 18d includes graph 1812 that shows dynamic response when LIG is exposed to large humidity stimulus. FIG. 18e includes graph 1814 that shows dynamic response when LIG is exposed to lower humidity stimulus. FIG. 18f includes a diagram 1816 that shows mechanism of temperature sensing. FIG. 18g includes a picture 1820 of a temperature sensor that includes an ecoflex layer 1822, LIG 1824, and a heat transfer paper 1826 on the textile 188. FIG. 18h includes a graph 1830 that illustrates a calibration plot. FIGS. 18i-j include graph 1832 and 1834, respectively that show dynamic response of an LIG-based temperature sensor upon contact with and removal from the human body.

Although specific LIG textiles and LIG textile considerations are discussed above with respect to FIGS. 14a-18j, any of a variety of LIG textiles and LIG textile considerations as appropriate to the requirements of a specific application can be utilized in accordance with embodiments of the invention. While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. A textile body area network, comprising:
a wireless reader;
a first smart textile configured to perform cross-body measurements, the first smart textile comprising:
a first array of magnetically coupled resonators configured to propagate magnetoinductive (MI) surface waves; and
at least one first sensor configured to connect to the first array of magnetically coupled resonators, wherein the at least one first sensor generates an output signal based on a physical measurement.

2. The textile body area network of claim 1, wherein:
the first array of magnetically coupled resonators comprises a first plurality of MI elements; and
the first array of magnetically coupled resonators creates a first flexible magnetic metamaterial path for wireless communication using the MI surface waves.

3. The textile body area network of claim 1, wherein the at least one first sensor is a laser-induced graphene sensor.

4. The textile body area network of claim 1, wherein the at least one first sensor is a strain sensor that measures motion and respiration.

5. The textile body area network of claim 1, wherein the at least one first sensor is a photoplethysmography (PPG) sensor that measures circulation, pulse transit, and blood pressure.

6. The textile body area network of claim 1, wherein the at least one first sensor is a bioelectricity sensor for electromyography (EMG) and electrocardiogram (ECG).

7. The textile body area network of claim 1, wherein the first smart textile is constructed by heat pressing the at least one first sensor to a first article of clothing, overlapping a textile system-patch with the at least one first sensor, and sewing the textile system-patch to the first article of clothing.

8. The textile body area network of claim 1, wherein the first smart textile is constructed by attaching the at least one first sensor to a textile system-patch and sewing the textile system-patch to a first article of clothing.

9. The textile body area network of claim 1, wherein the first smart textile is constructed by adhering the at least one first sensor to a first article of clothing and adhering a textile system-patch to the first article of clothing.

10. The textile body area network of claim 1, wherein the first smart textile is constructed using a compression strap and an external adjustable strap.

11. The textile body area network of claim 1 further comprising:
a second smart textile configured to perform cross-body measurements, the second smart textile comprising:
a second array of magnetically coupled resonators configured to propagate MI surface waves; and
at least one second sensor configured to connect to the second array of magnetically coupled resonators, wherein the at least one second sensor generates an output signal based on a physical measurement.

12. The textile body area network of claim 11, wherein:
the second array of magnetically coupled resonators comprises a second plurality of MI elements; and
the second array of magnetically coupled resonators creates a second flexible magnetic metamaterial path for wireless communication using the MI surface waves.

13. The textile body area network of claim 11, wherein the at least one second sensor is a laser-induced graphene sensor.

14. The textile body area network of claim 11, wherein the at least one second sensor is a strain sensor that measures motion and respiration.

15. The textile body area network of claim 11, wherein the at least one second sensor is a PPG sensor that measures circulation, pulse transit, and blood pressure.

16. The textile body area network of claim 11, wherein the at least one second sensor is a bioelectricity sensor for EMG and ECG.

17. The textile body area network of claim 11, wherein the second smart textile is constructed by heat pressing the at least one second sensor to a second article of clothing, overlapping a textile system-patch with the at least one second sensor, and sewing the textile system-patch to the second article of clothing.

18. The textile body area network of claim 11, wherein the second smart textile is constructed by attaching the at least one second sensor to a textile system-patch and sewing the textile system-patch to a second article of clothing.

19. The textile body area network of claim 11, wherein the second smart textile is constructed by adhering the at least one second sensor to a second article of clothing and adhering a textile system-patch to the second article of clothing.

20. The textile body area network of claim 11, wherein the second smart textile is constructed using a compression strap and an external adjustable strap.

21. The textile body area network of claim 11, wherein the first smart textile and the second smart textile are battery-free.

22. The textile body area network of claim 11, wherein the first article of clothing is a long shirt and the second article of clothing is a pants.

23. The textile body area network of claim 22, wherein the first smart textile and the second smart textile are separated by a clothing transition.

24. The textile body area network of claim 23, wherein the MI surface waves generate a wireless communication link across the clothing transition.

25. The textile body area network of claim 1, wherein the wireless reader performs edge-based AI for learning, identification, and power-handling decisions.
